SEQUENCE LISTING (26 sequences)

SEQ ID NO: 1 - RNA, 23 nt, Homo sapiens
uacccuguagaaccgaauuugug

SEQ ID NO: 2 - DNA, 23 bp, Artificial sequence
  Synthetic primer
cacaaattcggttctacagggta

SEQ ID NO: 3 - RNA, 22 nt, Homo sapiens
uagcuuaucagacugauguuga

SEQ ID NO: 4 - DNA, 22 bp, Artificial sequence
  Synthetic primer
tcaacatcagtctgataagcta

SEQ ID NO: 5 - RNA, 21 nt, Homo sapiens
aggcaagaugcuggcauagcu

SEQ ID NO: 6 - DNA, 21 bp, Artificial sequence
  Synthetic primer
agctatgccagcatcttgcct

SEQ ID NO: 7 - RNA, 22 nt, Homo sapiens
aacccguagauccgaucuugug

SEQ ID NO: 8 - DNA, 22 bp, Artificial sequence
  Synthetic primer
cacaagatcggatctacgggtt

SEQ ID NO: 9 - RNA, 22 nt, Homo sapiens
cagugcaaugaugaaagggcau

SEQ ID NO: 10 - DNA, 22 bp, Artificial sequence
  Synthetic primer
atgccctttcatcattgcactg

SEQ ID NO: 11 - RNA, 21 nt, Homo sapiens
cugaccuaugaauugacagcc

SEQ ID NO: 12 - DNA, 21 bp, Artificial sequence
  Synthetic primer
ggctgtcaattcataggtcag

SEQ ID NO: 13 - RNA, 20 nt, Homo sapiens
agagguauagggcaugggaa

SEQ ID NO: 14 - DNA, 20 bp, Artificial sequence
  Synthetic primer
ttcccatgccctatacctct

SEQ ID NO: 15 - RNA, 22 nt, Homo sapiens
cugugcgugugacagcggcuga

SEQ ID NO: 16 - DNA, 22 bp, Artificial sequence
  Synthetic primer
tcagccgctgtcacacgcacag

SEQ ID NO: 17 - RNA, 21 nt, Homo sapiens
gaacggcuucauacaggaguu

SEQ ID NO: 18 - DNA, 21 bp, Artificial sequence
  Synthetic primer
aactcctgtatgaagccgttc

SEQ ID NO: 19 - RNA, 22 nt, Homo sapiens
uuuguucguucggcucgcguga

SEQ ID NO: 20 - DNA, 22 bp, Artificial sequence
  Synthetic primer
tcacgcgagccgaacgaacaaa

SEQ ID NO: 21 - RNA, 22 nt, Homo sapiens
aagacgggaggaaagaagggag

SEQ ID NO: 22 - DNA, 22 bp, Artificial sequence
  Synthetic primer
ctcccttctttcctcccgtctt

SEQ ID NO: 23 - RNA, 22 nt, Homo sapiens
gucauacacggcucuccucucu

SEQ ID NO: 24 - DNA, 22 bp, Artificial sequence
  Synthetic primer
agagaggagagccgtgtatgac

SEQ ID NO: 25 - RNA, 23 nt, Homo sapiens
aaggagcuuacaaucuagcuggg

SEQ ID NO: 26 - DNA, 23 bp, Artificial sequence
  Synthetic primer
cccagctagattgtaagctcctt

(19) United States
(12) Patent Application Publication
(10) Pub. No.: US 2018/0327856 A1
(43) Pub. Date: Nov. 15, 2018

(54) DIAGNOSTIC MIRNAS FOR DIFFERENTIAL DIAGNOSIS OF INCIDENTAL PANCREATIC CYSTIC LESIONS

(71) Applicant: INTERPACE DIAGNOSTICS, LLC, Parsippany, NJ (US)

(72) Inventors: Dennis Wylie, Austin, TX (US); Anna E. Szafranska-Schwarzbach, Austin, TX (US); Bernard Andruss, Austin, TX (US); Alex Adai, Austin, TX (US); Linda S. Lee, Austin, TX (US); Darwin L. Conwell, Austin, TX (US)

(21) Appl. No.: 15/826,909

(22) Filed: Nov. 30, 2017

Related U.S. Application Data
(63) Continuation of application No. 13/801,737, filed on Mar. 13, 2013, now abandoned.
(60) Provisional application No. 61/716,396, filed on Oct. 19, 2012.
(60) Provisional application No. 61/709,411, filed on Oct. 4, 2012.

(51) Int. Cl. C12Q 1/6886

(52) U.S. Cl. CPC C12Q 1/6886; C12Q 2600/178; C12Q 2600/158; C12Q 2600/112

(57) ABSTRACT

Embodiments concern methods and compositions for characterizing or evaluating neoplastic pancreatic cells using miRNAs that are measured and used in calculations to determine a risk score for a patient.

This application claims priority to U.S. Provisional Patent Application 61/709,411 filed on Oct. 4, 2012 and U.S. Provisional Patent Application 61/716,396 filed Oct. 19, 2012, which are hereby incorporated by reference in their entirety.

### BACKGROUND OF THE INVENTION

### 1. Field of the Invention

The present invention relates generally to the field of medicine. Particularly, it concerns the use of biomarkers to distinguish benign from pre-malignant pancreatic cystic neoplasms and malignant pancreatic lesions.

### 2. Description of Related Art

With improvements in abdominal radiologic imaging, incidental pancreatic cystic neoplasms are increasingly discovered in as many as 20% of patients undergoing computed tomography (CT) scan or magnetic resonance imaging (MRI) of the abdomen for non-pancreatic indications. The differential diagnoses for incidental pancreatic cystic lesions include the following: 1) serous cystadcnoma (SN) or 2) pre-malignant mucinous cystic lesions, which are categorized into mucinous cystic neoplasm (MCN), branch-duct intraductal papillary mucinous neoplasm (BD-IPMN), and main-duct IPMN (MD-IPMN). Based on the histologic type, pancreatic cystic neoplasms have low or high risk for malignant transformation. Current guidelines from several major gastrointestinal societies recommend surgical resection for all definite MCNs and MD-IPMN since malignancy is detected in about 40-50% of resected MD-IPMN specimens. Because the occurrence of malignancy is much lower in BD-IPMN (15-20% of patients), resection is reserved for those patients with pancreatitis, cysts >3 cm, main pancreatic duct dilation greater than 10 mm, and/or the presence of mural nodules. Non-mucinous pancreatic lesions (serous cystadenomas) do not require further evaluation.

Differentiating and predicting malignant transformation in pancreatic cystic lesions is challenging. Current evaluation of suspicious pancreatic cystic neoplasms includes a combination of radiologic imaging, endoscopic ultrasound and cyst fluid analyses; however they are not adequate. For example, CT, MRI and EUS imaging features are only 50% accurate in diagnosing these lesions. Cytologic evaluation of aspirated fluid from fine needle aspiration (FNA) is often performed during EUS, but at best has a sensitivity of 34% for mucinous lesions. Carcinoembryonic antigen (CEA), secreted by epithelium lining mucinous lesions into cyst fluid, has modest sensitivity and specificity for mucinous cystic lesions of 75% and 84%, respectively (CEA >192 ng/mL) and is not predictive of malignancy. In addition, many mucinous lesions with CEA <192 ng/mL are missed using this cutoff. Measurement of allelic loss amplitude has a sensitivity of 67% and specificity of 66% for mucinous cystic lesions. The presence of K-ras mutation is highly specific (96%) for mucinous lesions, but has a low sensitivity of 45%. Therefore, current methodologies including imaging, endoscopy, and cyst fluid analysis fail to differentiate mucinous from non-mucinous pancreatic cystic lesions and cannot predict malignant transformation with a high degree of accuracy. New biomarkers for cystic lesions are needed to address current issues of diagnostic sensitivity and specificity.

### SUMMARY OF THE INVENTION

The disclosed methods and compositions overcome problems in the art by providing ways to use the expression of different miRNAs as biomarkers to characterize, identify, qualify, or distinguish between different types of neoplastic pancreatic cells, including but not limited to mucinous cystic neoplasm (MCN), serous cystadenoma (SC or also SN), pancreatic ductal adenocarcinoma (PDAC), intraductal papillary mucinous neoplasm (IPMN), or subtype of these. Embodiments concern differentiating between benign, pre-malignant, and malignant pancreatic lesions and cysts. This provides a clinician with information useful for diagnosis and/or for evaluating treatment options. It may also confirm an assessment based on the cytology of the patient's pancreas cells or on the patient's medical history or on the patient's symptoms or on some other test.

Further, methods are provided for diagnosing abnormal pancreatic cells based on determining expression levels of selected miRNAs in patient-derived samples that contain pancreatic cells. Additional methods provide information for evaluating pancreatic neoplastic cells in a biological sample from a patient.

Other embodiments concern methods for distinguishing benign from pre-malignant or malignant pancreatic cells in a biological sample from a patient suspected of having pancreatic neoplastic cells. Particular methods involve evaluating neoplastic pancreatic cells from a patient. Additional methods relate to distinguishing mucinous cystic neoplasm (MCN) pancreatic cells from intraductal papillary mucinous neoplasm (IPMN) pancreatic cells in a sample from a patient.

In some cases, methods pertain to distinguishing mucinous cystic neoplasm (MCN) pancreatic cells from other neoplastic pancreatic cells comprising SN, PDAC, or IPMN pancreatic cells in a sample from a patient. In other cases there are methods for distinguishing serous neoplastic pancreatic cells from other neoplastic pancreatic cells comprising MCN, PDAC, or IPMN pancreatic cells in a sample from a patient. Embodiments also include methods for distinguishing pancreatic ductal adenocarcinoma pancreatic cells from IPMN pancreatic cells in a sample from a patient.

In some embodiments, methods concern a patient who has already been determined to have pancreatic neoplastic cells or has been diagnosed as having pancreatic neoplastic cells. In other embodiments, a cytological examination or evaluation of the pancreatic cells has been done, is being done, or will be done on a sample comprising pancreatic cells. In certain cases, the cytology of a patient's pancreatic cells has already been evaluated and determined to be neoplastic or likely neoplastic. In certain embodiments, methods involve performing a cytological evaluation on a patient's pancreatic cells or confirming a cytology analysis that indicates that pancreatic cells are neoplastic.

Methods involve obtaining information about the levels of expression of certain microRNAs or miRNAs whose expression levels differ in different types of neoplastic pancreatic cells, such as cells from solid pancreatic tissue. In some embodiments, an evaluation of multiple differences in miRNA expression between or among different types of neoplastic pancreatic cells can be highly informative to a clinician. Such differences are highlighted when expression levels are first compared among two or more miRNAs and those differential values are compared to or contrasted with the differential values of a subset or subset of neoplastic pancreatic cells. Embodiments concern methods and compositions that can be used for evaluating pancreas cells or a pancreas sample, differentiating neoplastic pancreatic cells, distinguishing neoplastic pancreatic from one another, identifying a subtype of neoplastic pancreatic cells, identifying neoplastic pancreatic cells as a target for surgical resection, determining neoplastic pancreatic cells should not be surgically resected, categorizing abnormal pancreatic cells, diagnosing neoplastic pancreatic cells or diagnosing benign pancreas cells or diagnosing pre-malignant or malignant pancreatic cells, providing a prognosis to a patient regarding abnormal pancreatic cells or symptoms of one or more subtypes of neoplastic pancreatic cells, evaluating treatment options for neoplastic pancreatic pre-cancers or cancers, or treating a patient with MCN, PDAC, or IPMN. These methods can be implemented involving steps and compositions described below in different embodiments.

In some embodiments, methods involve determining from the biological sample the levels of expression of a plurality of miRNAs from the following group of biomarker miRNAs: miR-10b-5p, miR-21-5p, miR-31-5p, miR-99a-5p, miR-130b-3p, miR-192-5p, miR-202-3p, miR-210, miR-337-5p, miR-375, miR-483-5p, miR-485-3p, and miR-708-5p. Other embodiments, include the same plurality, but in some embodiments, the listing of the miRNAs is ordered as follows (most important miRNA first and remainder in decreasing order of importance): miR-202-3p, miR-483-5p, miR-31.5p, miR-192-5p, miR-708-5p, miR-21-5p, miR-375, miR-210, miR-99a-5p, miR-485-3p, miR-10b-5p, miR-337-5p, and, miR-130b-3p. In some embodiments, at least four of the listed miRNAs are used in methods. In certain other embodiments, methods involve determining from the biological sample the levels of expression of a miR-202-3p, miR-483-5p, miR-31-5p, and miR-192-5p.

A plurality means more than 1 and in the context of such methods, the expression levels of 2, 3, 4, 5, 6, 7, 8, 9, 10, 11, 12, or all 13 miRNAs (or any range derivable therein) may be determined. Methods may further involve calculating a risk score for the biological sample that identifies the sample as containing pancreatic cells that are characterized as mucinous cystic neoplasm (MCN), serous cystadenoma (SN), pancreatic ductal adenocarcinoma (PDAC), intraductal papillary mucinous neoplasm (IPMN), or a subtype thereof.

It will be understood that “determining the level of expression” refers to measuring or assaying for expression of the recited microRNA using a probe that is at least 98% complementary to the entire length of the mature human miRNA sequence, which will involve performing one or more chemical reactions. In some embodiments, a probe that is at least 99% or 100% complementary to the sequence of the entire length of the most predominant mature human miRNA sequence is used to implement embodiments discussed herein. In other embodiments a probe that is at least 99% or 100% complementary to the sequence of the entire length of the cDNA copy of the most predominant mature human mrRNA sequence is used to implement embodiments discussed herein. it is contemplated that while additional miRNAs that are nearly identical to the recited miRNA may be measured in embodiments, the recited miRNA whose expression is being evaluated is at least one of the miRNAs whose expression is being measured in embodiments. These different recited human miRNA sequences are provided in SEQ ID NOs: 1, 3, 5, 7, 9, 11, 13, 15, 17, 19, 21, 23, or 25. Mature miRNAs may be indirectly determined by directly measuring precursor microRNA molecules; in some embodiments, this is done using the same probe that is used for measuring mature miRNAs. In some embodiments, the amount or value of the expression level may be obtained or provided, which means it is made known (and determined beforehand). Embodiments involve 1, 2, 3, 4, 5 6, 7, 8, 9, 10, 11, 12, or 13 or more probes that are at least 90, 91, 92, 93, 94, 95, 96, 97, 98, 99. or 100% identical to SEQ ID NO:2, SEQ ID NO:4. SEQ ID NO:6. SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 or SEQ ID NO:26, depending on which miRNA is being measured. Alternatively, the probes could be used to detect a cDNA copy of the miRNA in question. In some cases, embodiments involving probe detection may involve 1, 2, 3, 4, 5 6, 7, 8, 9, 10, 11, 12, or 13 or more probes that are at least 90, 91, 92, 93, 94, 95, 96, 97, 98, 99, or 100% identical to SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23 or SEQ ID NO:25, depending on which miRNA is being measured.

Some embodiments involve determining from the biological sample the levels of expression of a plurality of at least four miRNAs from the following group of biomarker miRNAs: miR-10b-5p, miR-21-5p, miR-31-5p, miR-99a-5p, miR-130b-3p, miR-192-5p, miR-202-3p, miR-210, miR-337-5p, miR-375, miR-483-5p, miR-485-3p, and miR-708-5p; and, calculating a risk score for the biological sample that identifies the sample as containing pancreatic cells that are characterized as benign, pre-malignant or malignant, 4, 5, 6, 7, 8, 9, 10, 11, 12, or all 13 miRNAs (or any range derivable therein) may have their level of expression determined.

Some other embodiments also involve determining from the sample the levels of expression of at least the following miRNAs: miR-130b-3p, miR-192-5p, miR-202-3p, and miR-337-5p. and, calculating a risk score for the biological sample that identifies the sample as containing pancreatic cells that are characterized as MCN or IPMN, or a subtype thereof. In certain embodiments, the expression level of miR-202-3p is weighted more heavily than the expression levels of miR-130b-3p, miR-192-5p and miR-337-5p in calculating the risk score.

In particular embodiments, there are methods involving determining from the sample the levels of expression of at least the following miRNAs: miR-202-3p, miR-210, and miR-375; and, calculating a risk score for the biological sample that identifies the sample as containing pancreatic cells that are characterized as MCN or not MCN neoplastic pancreatic cells. In some embodiments, wherein the expression level of miR-202-3p is weighted more heavily than the expression levels of miR-210, miR-202-3p and miR-375 in calculating the risk score.

In additional embodiments, methods involve determining from the sample the levels of expression of at least the following miRNAs: miR-3-5p, miR-99a-5p, miR-375, and miR-483-5p; and calculating a risk score for the biological sample that identifies the sample as containing pancreatic cells that are characterized as MCN or not MCN neoplastic pancreatic cells. In certain cases, the expression level of miR-483-5p is weighted more heavily than the expression levels of miR-99a-5p, miR-375, and miR-31-5p in calculating the risk score.

Other methods include distinguishing pancreatic ductal adenocarcinoma pancreatic cells from IPMN pancreatic cells in a sample from a patient by determining from the sample the levels of expression of at least the following miRNAs: miR-21-5p, miR-375, miR-485-3p, and miR-708-5p; and, by calculating a risk score for the biological sample that identifies the sample as containing pancreatic cells that are characterized as MCN or not MCN neoplastic pancreatic cells. In particular applications, wherein the expression level of miR-375 is weighted more heavily than the expression levels of miR-21-p, miR-485-3p, and miR-708-5p in calculating the risk score.

The weight of a particular expression level (or the value of that expression level) reflects its importance in the accuracy, specificity, integrity, or other parameter relating to quality, of the test. This can be implemented in the algorithm or reflected in a model coefficient. A person of ordinary skill in the art would know how to determine this based on the experimental data. In certain embodiments, weighing a value more heavily may involve adding or multiplying the value by a particular number such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000, or any range derivable therein.

Certain methods include measuring the level of expression in the neoplastic pancreatic cells of at least one of the following diff pair miRNAs: miR-10b-5p, miR-21-5p, miR-31-5p, miR-98, miR-125-3p, miR-130b-3p, miR-134, miR-135a-5p, miR-135b-5p, miR-192-5p, miR-194-5p, miR-200a-3p, miR-200b-3p, miR-200c-3p, miR-202.3p, miR-203, miR-210, miR, 224-5p, miR-323-3p, miR-337-5p, miR-345-5p, miR-363-3p, miR-379-5p, miR-382-5p, miR-429, miR-483-5p, miR-485-3p, miR-485-5p, miR-489, miR-708-5p, or miR-885-5p, wherein at least one of the miRNAs is a biomarker miRNA and one is a comparative miRNA; determining at least one biomarker diff pair value based on the level of expression of the biomarker miRNA compared to the level of expression of the comparative miRNA; and determining whether the neoplastic pancreatic cells are mucinous cystic neoplasm (MCN), serous cystadenoma (SN), pancreatic ductal adenocarcinoma (PDAC), intraductal papillary mucinous neoplasm (IPMN), or a subtype thereof, based on the biomarker diff pair value(s). Any other diff pair identified in the examples may be used in embodiments discussed herein. However, a person of ordinary skill in the art understands that different pair analysis factors may be used, particular with respect to altering the reference miRNA in a pair without affecting the concept of the embodiments discussed herein.

The term “diff pair miRNA” refers to a miRNA that is one member of a pair of miRNAs where the expression level of one miRNA of the diff pair in a sample is compared to the expression level of the other miRNA of the diff pair in the same sample. The miRNA after the slash (/) is the reference or comparative miRNA. The expression levels of two diff pair miRNAs may be evaluated with respect to each other, i.e., compared, which includes but is not limited to subtracting, dividing, multiplying or adding values representing the expression levels of the two diff pair miRNAs. The term “biomarker miRNA” refers to a miRNA whose expression level is indicative of a particular disease or condition. A biomarker miRNA may be a diff pair miRNA in certain embodiments. As part of a diff pair, the level of expression of a biomarker miRNA may highlight or emphasize differences in miRNA expression between different populations, such from benign pancreatic cells and pre-malignant and/or malignant pancreatic cells. In some embodiments, when miRNA expression is different in a particular population relative to another population, differences between miRNA expression levels can be increased, highlighted, emphasized, or otherwise more readily observed in the context of a diff pair. It will be understood that the terms “diff pair miRNA,” “biomarker miRNA,” and “comparative miRNA” are used for convenience and that embodiments discussed herein may or may not refer to miRNAs using these terms. Regardless of whether the terms are used, the implementation of methods, kits, and other embodiments remains essentially the same. In further embodiments, methods involve comparing levels of expression of different miRNAs in the pancreatic sample to each other or to expression levels of other biomarkers, which occurs after a level of expression is measured or obtained. In certain embodiments, miRNA expression levels are compared to each other. In some embodiments, methods involve comparing the level of expression of the at least one biomarker miRNA to the level of expression of a comparative microRNA to determine a biomarker diff pair value. In some cases, methods may involve determining the level of 1, 2, 3, 4, 5, 6, 7, 8, 9, 10, II, 12, 13, 14, 15, 16, 17, 18, 19, 20, 21, 22, 23, 24, 25 or more diff pairs or any range derivable therein.

A “comparative miRNA” refers to a miRNA whose expression level is used to evaluate the level of another miRNA in the sample; in some embodiments, the expression level of a comparative microRNA is used to evaluate a biomarker miRNA expression level. For example, a differential value between the biomarker miRNA and the comparative miRNA can be calculated or determined or evaluated; this value is a number that is referred to as a “diff pair value” when it is based on the expression level of two miRNAs. A diff pair value can be calculated, determined or evaluated using one or more mathematical formulas or algorithms. In some embodiments, the value is calculated, determined or evaluated using computer software. Moreover, it is readily apparent that the miRNA used as a biomarker and the miRNA used as the comparative miRNA may be switched, and that any calculated value can be evaluated accordingly by a person of ordinary skill in the art. However, a person of ordinary skill in the art understands that different pair analysis may be adjusted, particular with respect to altering the comparative miRNA in a pair without affecting the concept of the embodiments discussed herein.

A comparative miRNA may be any miRNA, but in some embodiments, the comparative miRNA is chosen because it allows a statistically significant and/or relatively large difference in expression to be detected or highlighted between expression levels of the biomarker in one pancreatic cyst population as compared to a different pancreatic cyst population. Furthermore, a particular comparative miRNA in a diff pair may serve to increase any difference observed between diff pair values of different type of neoplastic pancreatic cells, for example, an MCN cell population compared to a IPMN or BD-IPMN cell population. In further embodiments, the comparative miRNA expression level serves as an internal control for expression levels. In some embodiments, the comparative miRNA is one that allows the relative or differential level of expression of a biomarker miRNA to be distinguishable from the relative or differential level of expression of that same biomarker in a different pancreatic cyst population. In some embodiments, the expression level of a comparative miRNA is a normalized level of expression for the different pancreatic cyst populations, while in other embodiments, the comparative miRNA level is not normalized. In some embodiments, there are methods for distinguishing or identifying pancreatic cancer cells in a patient comprising determining the level of expression of one or more miRNAs in a biological sample that contains pancreatic cells from the patient.

In some embodiments, methods will involve determining or calculating a diagnostic or risk score based on data concerning the expression level of one or more miRNAs, meaning that the expression level of the one or more miRNAs is at least one of the factors on which the score is based. A diagnostic or risk score will provide information about the biological sample, such as the general probability that the pancreatic sample contains premalignant or malignant cells or that the pancreatic sample does not contain such cells or has benign cells. In some embodiments, the diagnostic or risk score represents the probability that the patient is more likely than not to have a certain subtype of neoplastic pancreatic cells, such as PDAC, IPMN, SN, or MCN. In other embodiments, the diagnostic or risk score represents the probability that the patient has benign cells or non-malignant or nonpre-malignant cancer cells. In certain embodiments, a probability value is expressed as a numerical integer that represents a probability of 0% likelihood to 100% likelihood that a patient has a subtype or does not have that subtype (or has benign cells or has premalignant cells or has malignant cells). In some embodiments, the probability value is expressed as a numerical integer that represents a probability of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% likelihood (or any range derivable therein) that a patient has cells of a certain neoplastic pancreatic subtype (or a grouping of subtypes). In certain embodiments, multiple risk scores may be determined or calculated. In some embodiments, an aggregate risk score may be calculated that includes multiple risk scores that have been determined or calculated. There may be a risk score that reflects the general probability that a patient has 1) MCN versus other disease states (such as SN, IPMN and/or PDAC); 2) serous cystadenoma versus other disease states (such as MCN, IPMN, and/or PDAC); 3) IPMN from other disease states (such as MCN, PDAC, SN. and/or malignant lesions), and/or MCN versus other disease states (such as MCN, PDAC, SN, and/or malignant lesions). In certain embodiments a classifier is employed that involves multiple biomarkers and their statistics regarding the incidence of the biomarker and the level of expression compared either to one or more other biomarkers in a sample or to a reference level in order to calculate a risk score. The Examples provided herein show that this can be used successfully in the context of pancreatic conditions.

In a particular embodiment, there is a decision tree as follows: determine a risk score for MCN, and if the patient does not have a risk score indicative of MCN, determine a risk score for SN and/or PDAC. In certain embodiments, the decision tree involves determining the risk score for serous cystadenoma, and if the patient has a risk score that is not indicative of SN, the determinate a risk score for PDAC. In some cases, the decision tree further involves determining a risk score that compares the probability of having MCN versus IPMN, such as BD-IPMN.

In some embodiments, methods include evaluating one or more differential pair values using a scoring algorithm to generate a diagnostic or risk score for having PDAC, wherein the patient is identified as having or as not having such a based on the score. It is understood by those of skill in the art that the score is a predictive value about whether the patient does or does not have PDAC. In some embodiments, a report is generated and/or provided that identifies the diagnostic score or the values that factor into such a score. In some embodiments, a cut-offscore is employed to characterize a sample as likely having PDAC (or alternatively not having PDAC). In some embodiments, the risk score for the patient is compared to a cut-off score to characterize the biological sample from the patient with respect to whether they are likely to have or not to have PDAC.

In some embodiments, the sample comprises resected pancreatic tissue. In additional embodiments, methods involve obtaining the biological sample from the patient. In particular cases, methods may also involve doing a cytology analysis on the biological sample prior to determining expression levels of miRNAs. In some cases, the biological sample is formalin-fixed paraffin embedded (FFPE). In certain embodiments, a sample is first evaluated using cytology, and only if the sample is characterized as neoplastic by cytology is the sample then evaluated with respect to the level of expression of one or more miRNAs, as discussed herein. In some cases, if the sample is characterized as benign, pre-malignant, malignant or something other than non-neoplastic, then the sample is evaluated for miRNA expression levels. In particular embodiments, the sample comprises cystic fluid. In certain cases, cystic fluid is obtained from a fine needle aspirate. The cystic fluid may or may not contain cells from the cyst wall. It is contemplated that any embodiment discussed herein with respect to an FFPE sample, may be implemented with a sample comprising cystic fluid.

Embodiments concern characterizing neoplastic pancreatic tissue. In some embodiments, the characterization is provided as a probability that the patient has a particular type of neoplastic pancreatic cells or tissue. Accordingly, some methods and embodiments, include calculating a risk score. In some embodiments, involves calculating a risk score using a computer and an algorithm. In specific embodiments, calculating a risk score comprises applying model coefficients to each of the levels of expression. Model coefficients may be determined using logistic regression modeling, linear discriminant analysis, quadratic discriminant analysis, neural network, support vector machine, k-nearest neighbor classifier, or a variation thereof. In certain embodiments, a logistic regression modeling is used to calculate 1, 2, 3, 4, 5, 6, 7, 8, 9, 10, 11, 12, or 13 model coefficients, or any range derivable therein. It is contemplated that there is a model coefficient that is determined and applied to a specific miR and its expression level value.

In further embodiments, methods also involve assaying or determining levels of other biological molecules. In some embodiments, methods comprise determining a level of amylase, CA 19-9, and/or carcinoembryonic antigen (CEA) in the patient. In other embodiments, a biological sample may be assayed for one or more mutations. In some cases, a sample is assayed for 1, 2, 3, 4, 5, 6, 7, 8, 9, 10, 12, 13, 14, 15 or more mutations (and any range derivable therein) in KRAS and/or GNAS. It is specifically contemplated that mutations in codons 12 and/or 13 in KRAS may be part of methods and other embodiments described herein.

In some embodiments, the plurality of biomarker miRNAs whose expression is determined comprises or consists of: miR-375; miR-202-3p; miR-130b-3p, miR-192-59, miR-202-3p, and miR-337-5p; miR-10b-5p, miR-202-3p, miR-210, and miR-375; miR-31-5p, miR-99a-5p, miR-375, and miR-483-5p; and/or miR-21-5p, miR-375, miR-485-3p, and miR-708-5p.

In some instances, embodiments include identifying the patient as having a risk score indicative of at least about, at most about, or equal to about a 10, 15, 20, 25, 30, 35, 40, 45, 50, 55, 60, 65, 70, 80, 85, 90, 95, 96, 97, 98, 99, 100% (or any range derivable therein) chance or greater of having a particular subtype of pancreatic neoplasm. Generally, any probably metric may be employed, including one that identifies a number between 0.0 and 1.0, which reflects the percent chance that a patient has the particular type of neoplastic pancreatic cell. For example, the risk score may use the following numbers: 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0. Therefore, in some embodiments these numbers may be used for identifying a patient as having a particular risk score, for example, of having IPMN (or a subtype), PDAC, or SN.

In further embodiments, methods involve resecting all or part of the pancreatic neoplastic cells from the patient. In other embodiments, the patient may be treated for a pancreatic lesion determined to be pre-malignant or malignant. In some instances, the patient is treated with chemotherapy and/or radiation.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor. Individual miRNAs have been identified and sequenced in different organisms, and they have been given names. Names of miRNAs that are related to the disclosed methods and compositions, as well as their sequences, are provided herein. The name of the miRNAs that are used in methods and compositions refers to an miRNA that is at least 90% identical to the named miRNA based on its matured sequence listed herein and that is capable of being detected under the conditions described herein using the designated ABI part number for the probe. In most embodiments, the sequence provided herein is the sequence that is being measured in methods described herein.

The term “naturally occurring” refers to something found in an organism without any intervention by a person; it could refer to a naturally-occurring wildtype or mutant molecule. In some embodiments a synthetic miRNA molecule, such as aprobe or primer, does not have the sequence of a naturally occurring miRNA molecule. In other embodiments, a synthetic miRNA molecule may have the sequence of a naturally occurring miRNA molecule, but the chemical structure of the molecule that is unrelated specifically to the precise sequence (i.e., non-sequence chemical structure) differs from chemical structure of the naturally occurring miRNA molecule with that sequence. Corresponding miRNA sequences that can be used in the context of the disclosed methods and compositions include, but are not limited to, all or a portion of those sequences in the SEQ ID NOs disclosed herein, as well as any other miRNA sequence, miRNA precursor sequence, or any sequence complementary thereof. In some embodiments, the sequence is or is derived from or contains all or part of a sequence identified herein to target a particular miRNA (or set of miRNAs) that can be used with that sequence.

Any of the methods described herein may be implemented on tangible computer-readable medium comprising computer-readable code that, when executed by a computer, causes the computer to perform operations one or more operations. In some embodiments, there is a tangible computer-readable medium comprising computer-readable code that, when executed by a computer, causes the computer to perform operations comprising: receiving information corresponding to a level of miRNA expression in a pancreatic sample from a patient comprising: miR-10b-5p, miR-21-5p, miR-31-5p, miR-99a-5p, miR-130b-3p, miR-192-5p, miR-202-3p, miR-210, miR-337-5p, miR-375, miR-483-5p, miR-485-3p, and miR-708-5p and calculating a risk score for the biological sample that identifies the sample as containing pancreatic cells that are characterized as mucinous cystic neoplasm (MCN), serous cystadenoma (SN), pancreatic ductal adenocarcinoma (PDAC), intraductal papillary mucinous neoplasm (IPMN), or a subtype thereof. In some embodiments the tangible computer-readable medium calculates a risk score using a computer and an algorithm. In yet other embodiments the tangible computer-readable medium calculates a risk score by applying model coefficients to each of the levels of expression of miRNAs measured. In still other embodiments the tangible computer-readable medium calculates a risk score by applying model coefficients and the model coefficients are determined using logistic regression modeling, linear discriminant analysis, quadratic discriminant analysis, neural network, support vector machine, k-nearest neighbor classifier, or a variation thereof. In some embodiments receiving information comprises receiving information corresponding to a level of expression in a pancreatic sample from a patient comprising: miR-10b-5p, miR-21-5p, miR-31-5p, miR-99a-5p, miR-130b-3p, miR-192-5p, miR-202-3p, miR-210, miR-337-5p, miR-375, miR-483-5p, miR-485-3p, and miR-708-5p, wherein at least one of the miRNAs is a biomarker miRNA. In still other embodiments, when computer-readable code is executed by a computer it causes the computer to perform one or more additional operations comprising sending information corresponding to the miR expression values or model coefficient values to a tangible data storage device. In some embodiments, the computer readable code is executed by a computer and causes the computer to perform one or more additional operations comprising sending information corresponding to the calculated risk score value to a tangible data storage device. In yet other embodiments the computer readable code is executed by a computer and causes the computer to perform one or more additional operations comprising sending information to a tangible data storage device information comprising: miR-10b-5p, miR-21-5p, miR-31-5p, miR-99a-5p, miR-130b-3p, miR-192-5p, miR-202-3p, miR-210, miR-337-5p, miR-375, miR-483-55p, miR-485-3p, and miR-708-5p, wherein at least one of the miRNAs is a biomarker miRNA. In some aspects the tangible computer-readable medium contains computer-readable code that, when executed by a computer, causes the computer to perform operations further comprising calculating a risk score for the pancreatic sample, wherein the risk score is indicative of the probability that the pancreatic sample contains mucinous cystic neoplasm (MCN), serous cystadenoma (SN), pancreatic ductal adenocarcinoma (PDAC). intraductal papillary mucinous neoplasm (IPMN), or a subtype thereof.

Any of the methods described herein may be implemented on tangible computer-readable medium comprising computer-readable code that, when executed by a computer, causes the computer to perform operations one or more operations. In some embodiments the computer-readable code, when executed by a computer, causes the computer to perform operations comprising receiving information corresponding to a level of miRNA expression in a pancreatic sample from a patient comprising at least one of the following diff pair miRNAs: miR-10b-5p, miR-21-5p, miR-3 i-5p, miR-9K, miR-125-3p, miR-130b-3p, miR-134, miR-135a-5p, miR-135b-5p, miR-192-5p, miR-194-5p, miR-200a-3p, miR-200b-3p, miR-200c-3p, miR-202-3p, miR-203, miR-210, miR-224-5p, miR-323-3p, miR-337-5p, miR-345-5p, miR-363-3p, miR-379-5p, miR-382-5p, miR-429, miR-483-5p, miR-485-3p, miR-485-5p, miR-489, miR-708-5p, or miR-885-5p, wherein at least one of the miRNAs is a biomarker miRNA and one is a comparative miRNA and determining at least one biomarker diff pair value based on the level of expression of the biomarker miRNA compared to the level of expression of the comparative miRNA and determining whether the neoplastic pancreatic cells are mucinous cystic neoplasm (MCN), serous neoplasm (SN), pancreatic ductal adenocarcinoma (PDAC), intraductal papillary mucinous neoplasm (IPMN), or a subtype thereof, based on the biomarker diff pair value(s). In still other embodiments the computer performs operations comprising receiving information, wherein the receiving information comprises receiving from a tangible data storage device information corresponding to a level of expression in a pancreatic sample from a patient comprising at least one of the following diff pair miRNAs: miR-10b-5p, miR-21-5p, miR-31-5p, miR-98, miR-125-3p, miR-130b-3, miR-134, miR-135a-5p, miR-135b-5p, miR-192-5p, miR-194-5p, miR-200a-3p, miR-200b-3p, miR-200c-3p, miR-202-3p, miR-203, miR-210, miR-224-5p, miR-323-3p, miR-337-5p, miR-345-5p, miR-363-3p, miR-379-5p, miR-382-5p, miR-429, miR-483-5p, miR-485-3p, miR-485-5p, miR-489, miR-708-5p, or miR-885-5p. In yet other embodiments the computer-readable code, when executed by a computer, causes the computer to perform one or more additional operations comprising sending information corresponding to the miR diff pair values to a tangible data storage device. In yet other embodiments the computer-readable code, when executed by a computer causes information to be sent to a tangible data storage device comprising at least one of the following diff pair miRNAs: miR-10b-5p, miR-21-5p, miR-31-5p, miR-98, miR-125-3p, miR-130b-3p, miR-134, miR-135a-5p, miR-135b-5p, miR-192-5p, miR-194-5p, miR-200a-3p, miR-200b-3p, miR-200c-3p, miR-202-3p, miR-203, miR-210, miR-224-5p, miR-323-3p, miR-337-5p, miR-345-5p, miR-363-3p, miR-379-5p, miR-382-5p, miR-429, miR-483-5p, miR-485-3p, miR-485-5p, miR-489, miR-708-5p, or miR-885-5p., wherein at least one of the miRNAs is a biomarker miRNA. In some aspects of the invention, the computer-readable code, when executed by a computer, causes the computer to perform operations further comprising calculating a risk score for the pancreatic sample, wherein the risk score is indicative of the probability that the pancreatic sample contains mucinous cystic neoplasm (MCN), serous neoplasm (SN), pancreatic ductal adenocarcinoma (PDAC), intraductal papillary mucinous neoplasm (IPMN), or a subtype thereof.

A processor or processors can be used in performance of the operations driven by the example tangible computer-readable media disclosed herein. Alternatively, the processor or processors can perform those operations under hardware control, or under a combination of hardware and software control. For example, the processor may be a processor specifically configured to carry out one or more those operations, such as an application specific integrated circuit (ASIC) or a field programmable gate array (FPGA). The use of a processor or processors allows for the processing of information (e.g., data) that is not possible without the aid of a processor or processors, or at least not at the speed achievable with a processor or processors. Some embodiments of the performance of such operations may be achieved within a certain amount of time, such as an amount of time less than what it would take to perform the operations without the use of a computer system, processor, or processors, including no more than one hour, no more than 30 minutes, no more than 15 minutes, no more than 10 minutes, no more than one minute, no more than one second, and no more than every time interval in seconds between one second and one hour.

Some embodiments of the present tangible computer-readable media may be, for example, a CD-ROM, a DVD-ROM, a flash drive, a hard drive, or any other physical storage device. Some embodiments of the present methods may include recording a tangible computer-readable medium with computer-readable code that, when executed by a computer, causes the computer to perform any of the operations discussed herein, including those associated with the present tangible computer-readable media. Recording the tangible computer-readable medium may include, for example, burning data onto a CD-ROM or a DVD-ROM, or otherwise populating a physical storage device with the data. Expression data, diff pair values, scaling matrix values, and/or risk scores may be stored or processed according to embodiments discussed herein.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. PCA Plot of the first two principal components of 35 miRNA singleplex PCR data set indicates that these miRNA can separate pancreatic samples by diagnostic grouping.

FIG. 2.A. MegaPlex DiffPairs: Strip plots of the MegaPlex-assayed expression (delta-Ct) values for the 30 differentially expressed DiffPairs from which miRNA candidates for further investigation by singleplex RT-qPCR were identified. B. MegaPlex miRs: Strip plots of the expression (Ct) values for 34 of the 35 miRNA candidates for singleplex PCR. Candidate miR-30a-3p is excluded from this plot because it was not run on MegaPlex (identified instead from prior research).

FIG. 3. Singleplex miRs: Strip plots of the singleplex-assayed expression (Ct) values for the 35 miRNA candidates.

### DESCRIPTION OF ILLUSTRATIVE EMBODIMENTS

Certain embodiments at directed to compositions and methods relating to preparation and characterization of miRNAs, as well as use of miRNAs for therapeutic, prognostic, and diagnostic applications, particularly those methods and compostions related to assessing and/or identifying pancreatic disease.

### I. miRNA Molecules

MicroRNA molecules (“miRNAs”, “miR”, “miRs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two region of complementarity that enable them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene. Examples of animal miRNAs include those that imperfectly basepair with the target, which halts translation of the target (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through an RNA-induced silencing complex (RISC) (Denli et al., 2003).

Examples of miRNA molecules, their sequences and probes that might be used to detect these are given in Table 8.

TABLE 8
Mature miRNA sequences

| miR name | Assay ID | Mature miRNA Sequence (5′-3′) | miRNA probe (5′-3′) |
|---|---|---|---|
| miR-10b-5p | 002218 | UACCCUGUAGAACCGAAUUUGUG | CACAAATTCGGTTCTACAGGGTA |
| | | (SEQ ID NO 1) | (SEQ ID NO 2) |
| miR-21-5p | 000397 | UAGCUUAUCAGACUGAUGUUGA | TCAACATCAGTCTGATAAGCTA |
| | | (SEQ ID NO 3) | (SEQ ID NO 4) |
| miR-31-5p | 002279 | AGGCAAGAUGCUGGCAUAGCU | AGCTATGCCAGCATCTTGCCT |
| | | (SEQ ID NO 5) | (SEQ ID NO 6) |
| miR-99a-5p | 000435 | AACCCGUAGAUCCGAUCUUGUG | CACAAGATCGGATCTACGGGTT |
| | | (SEQ ID NO 7) | (SEQ ID NO 8) |
| miR-130b-5p | 000456 | CAGUGCAAUGAUGAAAGGGCAU | ATGCCCTTTCATCATTGCACTG |
| | | (SEQ ID NO 9) | (SEQ ID NO 10) |
| miR-192-5p | 000491 | CUGACCUAUGAAUUGACAGCC | GGCTGTCAATTCATAGGTCAG |
| | | (SEQ ID NO 11) | (SEQ ID NO 12) |
| miR-202-5p | 002363 | AGAGGUAUAGGGCAUGGGAA | TTCCCATGCCCTATACCTCT |
| | | (SEQ ID NO 13) | (SEQ ID NO 14) |
| miR-210 | 000512 | CUGUGCGUGUGACAGCGGCUGA | TCAGCCGCTGTCACACGCACAG |
| | | (SEQ ID NO 15) | (SEQ ID NO 16) |
| miR-337-5p | 002156 | GAACGGCUUCAUACAGGAGUU | AACTCCTGTATGAAGCCGTTC |
| | | (SEQ ID NO 17) | (SEQ ID NO 18) |
| miR-375 | 000564 | UUUGUUCGUUCGGCUCGCGUGA | TCACGCGAGCCGAACGAACAAA |
| | | (SEQ ID NO 19) | (SEQ ID NO 20) |
| miR-483-5p | 002338 | AAGACGGGAGGAAAGAAGGGAG | CTCCCTTCTTTCCTCCCGTCTT |
| | | (SEQ ID NO 21) | (SEQ ID NO 22) |
| miR-485-5p | 001277 | GUCAUACACGGCUCUCCUCUCU | AGAGAGGAGAGCCGTGTATGAC |
| | | (SEQ ID NO 23) | (SEQ ID NO 24) |
| miR-708-5p | 002341 | AAGGAGCUUACAAUCUAGCUGGG | CCCAGCTAGATTGTAAGCTCCTT |
| | | (SEQ ID NO 25) | (SEQ ID NO 26) |

A. Nucleic Acids

In the disclosed compositions and methods miRNAs can be labeled, used in array analysis, or employed in diagnostic, therapeutic, or prognostic applications, particularly those related to pathological conditions of the pancreas. The RNA may have been endogenously produced by a cell, or been synthesized or produced chemically or recombinantly. They may be isolated and/or purified. The term “miR NA,” unless otherwise indicated, refers to the processed RNA, after it has been cleaved from its precursor. The name of the miRNA is often abbreviated and referred to without a hsa-, mmu-, or mo- prefix and will be understood as such, depending on the context. Unless otherwise indicated, miRNAs referred to are human sequences identified as miR-X or let-X, where X is a number and/or letter.

In certain experiments, a miRNA probe designated by a suffix “5P” or “3P” can be used. “5P” indicates that the mature miRNA derives from the 5′ end of the precursor and a corresponding “3P” indicates that it derives from the 3′ end of the precursor, as described on the World Wide Web at sanger.ac.uk. Moreover, in some embodiments, a miRNA probe is used that does not correspond to a known human miRNA. It is contemplated that these non-human miRNA probes may be used in embodiments or that there may exist a human miRNA that is homologous to the non-human miRNA. While the methods and compositions are not limited to human miRNA, in certain embodiments, miRNA from human cells or a human biological sample is used or evaluated. In other embodiments, any mammalian miRNA or cell, biological sample, or preparation thereof may be employed.

In some embodiments, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, control nucleic acids, and other probes and primers. In many embodiments, miRNAs are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids used in methods and compositions disclosed herein may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30.31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000, or any range derivable therein, contiguous nucleotides. It is further understood that the length of complementarity within a precursor miRNA or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of the SEQ ID NOs disclosed herein. The commonly used name of the miRNA is given (with its identifying source in the prefix, for example, “hsa” for human sequences) and the processed miRNA sequence. Unless otherwise indicated, a miRNA without a prefix will be understood to refer to a human miRNA. A miRNA designated, for example, as miR-1-2 in the application will be understood to refer to hsa-miR-1-2. Moreover, a lowercase letter in the name of a miRNA may or may not be lowercase for example, hsa-mir-130b can also be referred to as miR-130B. In addition, miRNA sequences with a “mu” or “mmu” sequence will be understood to refer to a mouse miRNA and miRNA sequences with a “rno” sequence will be understood to refer to a rat miRNA. The term “miRNA probe” refers to a nucleic acid probe that can identify a particular miRNA or structurally related miRNAs.

It is understood that a miRNA is derived from genomic sequences or a gene. In this respect, the term “gene” is used for simplicity to refer to the genomic sequence encoding the precursor miRNA for a given miRNA. However, embodiments may involve genomic sequences of a miRNA that are involved in its expression, such as a promoter or other regulatory sequences.

The term “recombinant” generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary. miRNA probes or nucleic acids can include, can be, or can be at least 60, 65, 70, 75, 80, 85, 90, 95, 96, 97, 98, 99 or 100% complementary to their target.

As used herein, “hybridization”, “hybridizes” or “capable of hybridizing” is understood to mean the forming of a double or triple stranded molecule or a molecule with partial double or triple stranded nature. The term “anneal” is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein, “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s). the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of such include hybridization performed at about 0.15 M to about 0.9 M NaCl at a temperature range of about 20° C. to about 50° C. Of course, it is within the skill of one in the art to further modify the low or high stringency conditions to suite a particular application.

1. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those with a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

A nucleobase may be comprised in a nucleoside or nucleotide, using any chemical or natural synthesis method described herein or known to one of ordinary skill in the art. Such a nucleobase may be labeled or may be part of a molecule that is labeled and contains the nucleobase.

2. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

3. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

4. Nucleic Acid Analogs

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods disclosed herein. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside, and nucleotide analogs or derivatives are well known in the art, and have been described (see for example, Scheit, 1980, incorporated herein by reference).

Additional non-limiting examples of nucleosides, nucleotides, or nucleic acids comprising 5-carbon sugar and/or backbone moiety derivatives or analogs, include those in: U.S. Pat. No. 5,681,947, which describes oligonucleotides comprising purine derivatives that form triple helixes with and/or prevent expression of dsDNA; U.S. Pat. Nos. 5,652,099 and 5,763,167, which describe nucleic acids incorporating fluorescent analogs of nucleosides found in DNA or RNA, particularly for use as fluorescent nucleic acid probes; U.S. Pat. No. 5,614,617, which describes oligonucleotide analogs with substitutions on pyrimidine rings that possess enhanced nuclease stability; U.S. Pat. Nos. 5,670,663, 5,872,232 and 5,859,221, which describe oligonucleotide analogs with modified 5-carbon sugars (i.e., modified 2′-deoxyfuranosyl moieties) used in nucleic acid detection; U.S. Pat. No. 5,446,137, which describes oligonucleotides comprising at least one 5-carbon sugar moiety substituted at the 4′ position with a substituent other than hydrogen that can be used in hybridization assays; U.S. Pat. No. 5,886,165, which describes oligonucleotides with both deoxyribonucleotides with 3′-5′ internucleotide linkages and ribonucleotides with 2′-5′ internucleotide linkages; U.S. Pat. No. 5,714,606, which describes a modified internucleotide linkage wherein a 3′-position oxygen of the internucleotide linkage is replaced by a carbon to enhance the nuclease resistance of nucleic acids; U.S. Pat. No. 5,672,697, which describes oligonucleotides containing one or more 5′ methylene phosphonate internucleotide linkages that enhance nuclease resistance; U.S. Pat. Nos. 5,466,786 and 5,792,847, which describe the linkage of a substituent moiety which may comprise a drug or label to the 2′ carbon of an oligonucleotide to provide enhanced nuclease stability and ability to deliver drugs or detection moieties; U.S. Pat. No. 5,223,618, which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967, which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240, which describe oligonucleotides with three or four atom linker moiety replacing phosphodiester backbone moiety used for improved nuclease resistance, cellular uptake, and regulating RNA expression; U.S. Pat. No. 5,858,988, which describes hydrophobic carrier agent attached to the 2′-O position of oligonucleotides to enhanced their membrane permeability and stability; U.S. Pat. No. 5,214,136, which describes oligonucleotides conjugated to anthraquinone at the 5′ terminus that possess enhanced hybridization to DNA or RNA; enhanced stability to nucleases; U.S. Pat. No. 5,700,922, which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154, which describes RNA linked to a DNA to form a DNA-RNA hybrid; U.S. Pat. No. 5,728,525, which describes the labeling of nucleoside analogs with a universal fluorescent label.

Additional teachings for nucleoside analogs and nucleic acid analogs are U.S. Pat. No. 5,728,525, which describes nucleoside analogs that are end-labeled; U.S. Pat. Nos. 5,637,683, 6,251,666 (L-nucleotide substitutions), and U.S. Pat. No. 5,480,980 (7-deaza-2′deoxyguanosine nucleotides and nucleic acid analogs thereof).

5. Modified Nucleotides

Labeling methods and kits may use nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the methods and compositions are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in some embodiments are alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, etc. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in some embodiments. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

B. Preparation of Nucleic Acids

A nucleic acid may be made by any technique known to one of ordinary skill in the art, such as for example, chemical synthesis, enzymatic production, or biological production. It is specifically contemplated that miRNA probes are chemically synthesized.

In some embodiments, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and is specifically incorporated herein by reference. Generally, methods involve lysing cells with a solution having guanidinium and a detergent.

Alternatively, nucleic acid synthesis is performed according to standard methods. See, for example, Itakura and Riggs (1980). Additionally, U.S. Pat. Nos. 4,704,362, 5,221,619, and 5,583,013 each describe various methods of preparing synthetic nucleic acids. Non-limiting examples of a synthetic nucleic acid (e.g., a synthetic oligonucleotide) include a nucleic acid made by in vitro chemical synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In some methods, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. Nos. 4,683,202 and 4,682,195, each incorporated herein by reference), or the synthesis of an oligonucleotide as described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Basically, chemical synthesis can be achieved by the diester method, the triester method, polynucleotide phosphorylase method and by solid-phase chemistry. The diester method was the first to be developed to a usable state, primarily by Khorana and co-workers. (Khorana, 1979). The basic step is the joining of two suitably protected deoxynucleotides to form a dideoxynucleotide containing a phosphodiester bond.

The main difference between the diester and triester methods is the presence in the latter of an extra protecting group on the phosphate atoms of the reactants and products (Itakura et al., 1975). Purifications are typically done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method is an enzymatic method of DNA synthesis that can be used to synthesize many useful oligonucleotides (Gillam et al., 1978; Gillam et al., 1979). Under controlled conditions, polynucleotide phosphorylase adds predominantly a single nucleotide to a short oligonucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-phase methods draw on technology developed for the solid-phase synthesis of polypeptides. It has been possible to attach the initial nucleotide to solid support material and proceed with the stepwise addition of nucleotides. All mixing and washing steps are simplified, and the procedure becomes amenable to automation. These syntheses are now routinely carried out using automatic nucleic acid synthesizers.

Phosphoramidite chemistry (Beaucage and Lyer, 1992) has become the most widely used coupling chemistry for the synthesis of oligonucleotides. Phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

In certain embodiments, nucleic acid molecules are not synthetic. In some embodiments, the nucleic acid molecule has a chemical structure of a naturally occurring nucleic acid and a sequence of a naturally occurring nucleic acid, such as the exact and entire sequence of a single stranded primary miRNA (see Lee 2002), a single-stranded precursor miRNA, or a single-stranded mature miRNA. In addition to the use of recombinant technology, such non-synthetic nucleic acids may be generated chemically, such as by employing technology used for creating oligonucleotides.

C. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased.

Methods may involve the use of organic solvents and/or alcohol to isolate nucleic acids, particularly miRNA used in methods and compositions disclosed herein. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns. which may include a mineral or polymer support with electronegative groups. A glass fiber filter or column may work particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for forming a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried down and resuspended in a liquid and volume appropriate for subsequent manipulation.

As discussed above, some embodiments concern the detection of miRNA. The method may involve the conversion of RNA to complementary DNA (cDNA). The methods of converting RNA to cDNA are well known to those of skill in the art.

### II. Labels and Labeling Techniques

In some embodiments, miRNAs are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In particular embodiments, the label is non-radioactive. Generally, nucleic acids may be labeled by adding labeled nucleotides (one-step process) or adding nucleotides and labeling the added nucleotides (two-step process).

A. Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide(s) is catalytically added to a miRNA, and the unlabeled nucleotide is modified with a chemical moiety that enables it to be subsequently labeled. In some embodiments, the chemical moiety is a reactive amine such that the nucleotide is an amine-modified nucleotide. Examples of amine-modified nucleotides are well known to those of skill in the art, many being commercially available.

In contrast to labeling of cDNA during its synthesis, the issue for labeling miRNA is how to label the already existing molecule. Some aspects concern the use of an enzyme capable of using a di- or tri-phosphate ribonucleotide or deoxyribonucleotide as a substrate for its addition to a miRNA. Moreover, in specific embodiments, a modified di- or tri-phosphate ribonucleotide is added to the 3′ end of a miRNA. The source of the enzyme is not limiting. Examples of sources for the enzymes include yeast, gram-negative bacteria such as E. coli, lactococcus lactis. and sheep pox virus.

Enzymes capable of adding such nucleotides include, but are not limited to, poly(A) polymerase, terminal transferase, and polynucleotide phosphorylase. In specific embodiments, a ligase is contemplated as not being the enzyme used to add the label, and instead, a non-ligase enzyme is employed.

Terminal transferase may catalyze the addition of nucleotides to the 3′ terminus of a nucleic acid. Polynucleotide phosphorylase can polymerize nucleotide diphosphates without the need for a primer.

B. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include 1251, 32P, 33P, and 35S. Examples of enzymatic labels include alkaline phosphatase, luciferase, horseradish peroxidase, and β-galactosidase. Labels can also be proteins with luminescent properties, e.g., green fluorescent protein and phicoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer, and, TOTAB.

Specific examples of dyes include, but are not limited to, those identified above and the following: Alexa Fluor 350, Alexa Floor 405, Alexa Fluor 430, Alexa Fluor 488, Alexa Fluor 500. Alexa Fluor 514, Alexa Floor 532, Alexa Fluor 546, Alexa Floor 555, Alexa Fluor 568, Alexa Fluor 594, Alexa Fluor 610, Alexa Flouor 633, Alexa Flouor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2,4′,5′,7′-Tetrabromosulfonefluorescein, and TET.

Specific examples of fluorescently labeled ribonucleotides include Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides include Cy3-UTP and Cy5-UTP.

Examples of fluorescently labeled deoxyribonucleotides include Dinitrophenyl (DNP)-11-dUTP, Cascade Blue-?7-dUTP, Alexa Fluor 488-5-dUTP, Fluorescein-12-dUTP, Oregon Green 488-5-dUTP, BODIPY FL-14-dUTP, Rhodamine Green-S-dUTP, Alexa Floor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, and Alexa Fluor 647-12-OBEA-dCTP.

It is contemplated that nucleic acids may be labeled with two different labels. Furthermore, fluorescence resonance energy transfer (FRET) may be employed in disclosed methods (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and other ligands, include ligands for an antibody.

C. Visualization Techniques

A number of techniques for visualizing or detecting labeled nucleic acids are readily available. Such techniques include, microscopy, arrays, fluorometry, light cyclers or other real time PCR machines, FACS analysis, scintillation counters, phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996). spectroscopy; mass spectroscopy; radiological techniques; and mass balance techniques.

When two or more differentially colored labels are employed, fluorescent resonance energy transfer (FRET) techniques may be employed to characterize association of one or more nucleic acids. Furthermore, a person of ordinary skill in the art is well aware of ways of visualizing, identifying, and characterizing labeled nucleic acids, and accordingly, such protocols may be used. Examples of tools that may be used also include fluorescent microscopy, a BioAnalyzer, a plate reader, Storm (Molecular Dynamics), Array Scanner, FACS (fluorescence activated cell sorter), or any instrument that has the ability to excite and detect a fluorescent molecule.

### III. Array Preparation and Screening

A. Array Preparation

Some embodiments involve the preparation and use of miRNA arrays or miRNA probe arrays, which are ordered macroarrays or microarrays of nucleic acid molecules (probes) that are fully or nearly complementary or identical to a plurality of miRNA molecules or precursor miRNA molecules and that are positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass, metal, plastic, and silicon. Such arrays may vary in a number of different ways, including average probe length, sequence or types of probes, nature of bond between the probe and the array surface, e.g. covalent or non-covalent, and the like. The labeling and screening methods are not limited by with respect to any parameter except that the probes detect miRNA; consequently, methods and compositions may be used with a variety of different types of miRNA arrays.

Representative methods and apparatuses for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000, which are each herein incorporated by reference.

It is contemplated that the arrays can be high density arrays, such that they contain 2, 20, 25, 50, 80, 100, or more, or any integer derivable therein, different probes. It is contemplated that they may contain 1000, 16,000, 65,000, 250,000 or 1,000,000 or more, or any interger or range derivable therein, different probes. The probes can be directed to targets in one or more different organisms or cell types. In some embodiments, the oligonucleotide probes may range from 5 to 50, 5 to 45, 10 to 40, 9 to 34, or 15 to 40 nucleotides in length. In certain embodiments, the oligonucleotide probes are 5, 10, 15, 20, 25, 30, 35, 40 nucleotides in length, including all integers and ranges there between.

Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm2. The surface area of the array can be about or less than about 1, 1.6, 2, 3, 4, 5, 6, 7, 8, 9, or 10 cm2.

Moreover, a person of ordinary skill in the art could readily analyze data generated using an array. Such protocols are disclosed herein or may be found in, for example, WO 9743450; WO 03023058; WO 03022421; WO 03029485; WO 03067217; WO 03066906; WO 03076928; WO 03093810; WO 03100448A1, all of which are specifically incorporated by reference.

B. Sample Preparation

It is contemplated that the miRNA of a wide variety of samples can be analyzed using arrays, miRNA probes, or array technology. While endogenous miRNA is contemplated for use with compositions and methods disclosed herein, recombinant miRNA —including nucleic acids that are complementary or identical to endogenous miRNA or precursor miRNA—can also be handled and analyzed as described herein. Samples may be biological samples, in which case, they can be from biopsy, exfoliates, blood, tissue, organs, semen, saliva, tears, other bodily fluid, hair follicles, skin, or any sample containing or constituting biological cells. In certain embodiments, samples may be, but are not limited to, fresh, frozen, fixed, formalin fixed, paraffin embedded, or formalin fixed and paraffin embedded. Alternatively, the sample may not be a biological sample, but a chemical mixture, such as a cell-free reaction mixture (which may contain one or more biological enzymes).

Hybridization

After an array or a set of miRNA probes is prepared and the miRNA in the sample is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

C. Differential Expression Analyses

Arrays can be used to detect differences between two samples. Specifically contemplated applications include identifying and/or quantifying differences between miRNA from a sample that is normal and from a sample that is not normal, between a cancerous condition and a non-cancerous condition, or between two differently treated samples. Also, miRNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. These arrays may generally be produced using mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. No. 5,384,261, incorporated herein by reference in its entirety. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate (see U.S. Pat. Nos. 5,770,358, 5,789,162, 5,708,153, 6,040,193 and 5,800,992, each of which is hereby incorporated in its entirety). Arrays may be packaged in such a manner as to allow for diagnostics or other manipulation of an all inclusive device (see for example, U.S. Pat. Nos. 5,856,174 and 5,922,591, each incorporated in its entirety by reference). See also U.S. patent application Ser. No. 09/545,207, filed Apr. 7, 2000, which is incorporated by reference in its entirety for additional information concerning arrays, their manufacture, and their characteristics,

Moreover, miRNAs can be evaluated with respect to the following diseases, conditions, and disorders: pancreatitis, chronic pancreatitis, IPMN (and its subtypes), MCN and/or pancreatic cancer. Methods may also involve a distinct pancreatic tissue classifier disclosed in U.S. patent application Ser. No. 13/615,066 incorporated herein by reference.

Methods of the invention may also be used to detect or identify neoplastic pancreatic cysts including serous cystic tumors, mucinous cystic tumors, solid pseudopapillary tumors. Neoplastic pancreatic cysts detected or identified may further be subclassified as serous cystadenoma, serous cystadenocarcinoma, mucinous cystadenoma, mucinous cystadenoma with moderate dysplasia, infiltrating or noninfiltrating mucinous cystadenocarcinoma, intraductal papillary mucinous adenoma, intraductal papillary mucinous neoplasm with moderate dysplasia or infiltrating or noninfiltrating intraductal papillary mucinous carcinoma.

Cancers that may be evaluated by the disclosed methods and compositions include cancer cells particularly from the pancreas, including pancreatic ductal adenocarcinoma (PDAC), but may also include metastases to other organs such as liver, bone, lungs, brain, peritoneal cavity or lymphatic system. Moreover, miRNAs can be evaluated in precancers, such as metaplasia, dysplasia, and hyperplasia

Pancreatic metastases may also include but not are not limited to bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

It is specifically contemplated that the disclosed methods and compositions can be used to evaluate differences between stages of disease, such as between hyperplasia, neoplasia, pre-cancer and cancer, or between a primary tumor and a metastasized tumor.

Moreover, it is contemplated that samples that have differences in the activity of certain pathways may also be compared. These pathways include the following and those involving the following factors: antibody response, apoptosis, calcium/NFAT signaling, cell cycle, cell migration, cell adhesion, cell division, cytokines and cytokine receptors, drug metabolism, growth factors and growth factor receptors, inflammatory response, insulin signaling, NFκ-B signaling, angiogenesis, adipogenesis, cell adhesion, viral infection, bacterial infection, senescence, motility, glucose transport, stress response, oxidation, aging, telomere extension, telomere shortening, neural transmission, blood clotting, stem cell differentiation, G-Protein Coupled Receptor (GPCR) signaling, and p53 activation.

Cellular pathways that may be profiled also include but are not limited to the following: any adhesion or motility pathway including but not limited to those involving cyclic AMP, protein kinase A, G-protein couple receptors, adenylyl cyclase, L-selectin, E-selectin, PECAM, VCAM-1, α-actinin, paxillin, cadherins, AKT, integrin-α, integrin-β, RAF-1, ERK, PI-3 kinase, vinculin, matrix metalloproteinases, Rho GTPases, p85, trefoil factors, profilin, FAK, MAP kinase, Ras, caveolin, calpain-1, calpain-2, epidermal growth factor receptor, ICAM-1, ICAM-2, cofilin, actin, gelsolin, RhoA, RAC1, myosin light chain kinase, platelet-derived growth factor receptor or ezrin; any apoptosis pathway including but not limited to those involving AKT, Fas ligand, NFkappaB, caspase-9, PI3 kinase, caspase-3, caspase-7, ICAD, CAD, EndoG, Granzyme B, Bad, Bax, Bid, Bak, APAF-1, cytochrome C, p53, ATM, Bcl-2, PARP, Chk1, Chk2, p21, c-Jun, p73, Rad51, Mdm2, Rad50, c-Abl, BRCA-1, perforin, caspase-4, caspase-8, caspase-6, caspase-1, caspase-2, caspase-10, Rho, Jun kinase, Jun kinase kinase, Rip2, lamin-A, lamin-B1, lamin-B2, Fas receptor, H₂O₂, Granzyme A, NADPH oxidase, HMG2, CD4, CD28, CD3, TRADD, IKK, FADD, GADD45, DR3 death receptor, DR4/5 death receptor, FLIPs, APO-3, GRB2, SHC, ERK, MEK, RAF-1, cyclic AMP, protein kinase A, E2F, retinoblastoma protein, Smac/Diablo, ACH receptor, 14-3-3, FAK, SODD, TNF receptor, RIP, cyclin-DI, PCNA, Bcl-XL, PIP2, PIP3, PTEN, ATM, Cdc2, protein kinase C, calcineurin, IKKα, IKKβ, IKKγ, SOS-1, c-FOS, Traf-1, Traf-2, Iκββ or the proteasome; any cell activation pathway including but not limited to those involving protein kinase A, nitric oxide, caveolin-1, actin, calcium, protein kinase C, Cdc2, cyclin B, Cdc25, GRB2, SRC protein kinase, ADP-ribosylation factors (ARFs), phospholipase D, AKAP95, p68, Aurora B, CDK1, Eg7, histone H3, PKAc, CD80, PI3 kinase, WASP, Arp2, Arp3, p16, p34, p20, PP2A, angiotensin, angiotensin-converting enzyme, protease-activated receptor-1, protease-activated receptor-4, Ras, RAF-1, PLCβ, PLCγ, COX-1, G-protein-coupled receptors, phospholipase A2, IP3, SUMO1, SUMO 2/3, ubiquitin, Ran, Ran-GAP, Ran-GEF, p53, glucocorticoids, glucocorticoid receptor, components of the SWI/SNF complex, RanBP1, RanBP2, importins, exportins, RCC1, CD40, CD40 ligand, p38, IKKα, IKKβ, NFκB, TRAF2, TRAF3, TRAF5, TRAF6, IL-4, IL-4 receptor, CDK5, AP-1 transcription factor, CD45, CD4, T cell receptors, MAP kinase, nerve growth factor, nerve growth factor receptor, c-Jun, c-Fos, Jun kinase, GRB2, SOS-1, ERK-1, ERK, JAK2, STAT4, IL-12, IL-12 receptor, nitric oxide synthase, TYK2, IFNγ, elastase, IL-8, epithelins, IL-2, IL-2 receptor, CD28, SMAD3, SMAD4, TGFβ or TGFβ receptor, any cell cycle regulation, signaling or differentiation pathway including but not limited to those involving TNFs, SRC protein kinase, Cdc2, cyclin B, Grb2, Sos-1, SHC, p68, Aurora kinases, protein kinase A, protein kinase C, Eg7, p53, cyclins, cyclin-dependent kinases, neural growth factor, epidermal growth factor, retinoblastoma protein, ATF-2, ATM, ATR, AKT, CHK1, CHK2, 14-3-3, WEE1, CDC25 CDC6, Origin Recognition Complex proteins, p15, p16, p27, p21, ABL, c-ABL, SMADs, ubiquitin, SUMO, heat shock proteins, Wnt, GSK-3, angiotensin, p73 any PPAR, TGFα, TGFβ, p300, MDM2, GADD45, Notch, cdc34, BRCA-1, BRCA-2, SKP1, the proteasome, CUL1, E2F, p107, steroid hormones, steroid hormone receptors, IκBα, IκBβ, Sin3A, heat shock proteins, Ras, Rho, ERKs, IKKs, PI3 kinase, Bcl-2, Bax, PCNA, MAP kinases, dynein, RhoA, PKAc, cyclin AMP, FAK, PIP2, PIP3, integrins, thrombopoietin, Fas, Fas ligand, PLK3, MEKs, JAKs, STATs, acetylcholine, paxillin calcineurin, p38, importins, exportins, Ran, Rad50, Rad51, DNA polymerase, RNA polymerase, Ran-GAP, Ran-GEF, NuMA, Tpx2, RCC1, Sonic Hedgehog, Crm1, Patched (Pre-1), MPF, CaM kinases, tubulin, actin, kinetochore-associated proteins, centromere-binding proteins, telomerase, TERT, PP2A, c-MYC, insulin, T cell receptors, B cell receptors, CBP, IKβ, NFκB, RAC1, RAF1, EPO, diacylglycerol, c-Jun, c-Fos, Jun kinase, hypoxia-inducible factors, GATA4, β-catenin, α-catenin, calcium, arrestin, survivin, caspases, procaspases, CREB, CREM, cadherins, PECAMs, corticosteroids, colony-stimulating factors, calpains, adenylyl cyclase, growth factors, nitric oxide, transmembrane receptors, retinoids, G-proteins, ion channels, transcriptional activators, transcriptional coactivators, transcriptional repressors, interleukins, vitamins, interferons, transcriptional corepressors, the nuclear pore, nitrogen, toxins, proteolysis, or phosphorylation; or any metabolic pathway including but not limited to those involving the biosynthesis of amino acids, oxidation of fatty acids, biosynthesis of neurotransmitters and other cell signaling molecules, biosynthesis of polyamines, biosynthesis of lipids and sphingolipids, catabolism of amino acids and nutrients, nucleotide synthesis, eicosanoids, electron transport reactions, ER-associated degradation, glycolysis, fibrinolysis, formation of ketone bodies, formation of phagosomes, cholesterol metabolism, regulation of food intake, energy homeostasis, prothrombin activation, synthesis of lactose and other sugars, multi-drug resistance, biosynthesis of phosphatidylcholine, the protcasome, amyloid precursor protein, Rab GTPases, starch synthesis, glycosylation, synthesis of phoshoglycerides, vitamins, the citric acid cycle, IGF-1 receptor, the urea cycle, vesicular transport, or salvage pathways. It is further contemplated that the disclosed nucleic acids molecules can be employed in diagnostic and therapeutic methods with respect to any of the above pathways or factors. Thus, in some embodiments, a miRNA may be differentially expressed with respect to one or more of the above pathways or factors.

Phenotypic traits also include characteristics such as longevity, morbidity, appearance (e.g., baldness, obesity), strength, speed, endurance, fertility, susceptibility or receptivity to particular drugs or therapeutic treatments (drug efficacy), and risk of drug toxicity. Samples that differ in these phenotypic traits may also be evaluated using the arrays and methods described.

In certain embodiments, miRNA profiles may be generated to evaluate and correlate those profiles with pharmacokinetics. For example, miRNA profiles may be created and evaluated for patient tumor and blood samples prior to the patient being treated or during treatment to determine if there are miRNAs whose expression correlates with the outcome of the patient. Identification of differential miRNAs can lead to a diagnostic assay involving them that can be used to evaluate tumor and/or blood samples to determine what drug regimen the patient should be provided. In addition, identification of differential miRNAs can be used to identify or select patients suitable for a particular clinical trial. If a miRNA profile is determined to be correlated with drug efficacy or drug toxicity, such may be relevant to whether that patient is an appropriate patient for receiving a drug or for a particular dosage of a drug.

In addition to the above prognostic assays, blood samples from patients with a variety of diseases can be evaluated to determine if different diseases can be identified based on blood miRNA levels. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005, in the names of David Brown, Lance Ford, Angie Cheng and Rich Jarvis, which is hereby incorporated by reference in its entirety.

D. Other Assays

In addition to the use of arrays and microarrays, it is contemplated that a number of different assays could be employed to analyze miRNAs, their activities, and their effects. Such assays include, but are not limited to, nucleic acid amplification, polymerase chain reaction, quantitative PCR. RT-PCR, in situ hybridization, Northern hybridization, hybridization protection assay (HPA), branched DNA (bDNA) assay, rolling circle amplification (RCA), single molecule hybridization detection, Invader assay, and/or Bridge Litigation Assay.

E. Evaluation of Expression Levels and Diff Pair Values

A variety of different models can be employed to evaluate expression levels and/or other comparative values based on expression levels of miRNAs (or their precursors or targets). One model is a logistic regression model (see the Wikipedia entry on the World Wide Web at en.wikipedia.com, which is hereby incorporated by reference).

Start by computing the weighted sum of the DiffPair values:

z=β₀+β₁*Diff(miR₁ₐ,miR_{1b})+β₂*Diff(miR₂ₐ,miR_{2b})+ . . .

where the β₀ is the (Intercept) term identified in the spreadsheets, while the remaining β₁ are the weights corresponding to the various DiffPairs in the model in question. Once z is computed, the score p_{malignant} (which may be interpreted as predicted probability of malignancy) is calculated as

$${p}_{malignant} = \frac{1}{1 + exp ( - z )}$$

This functions to turn the number z, which may be any value from negative infinity to positive infinity, into a number between 0 and 1, with negative values for z becoming scores/probabilities of less than 50% and positive values for z becoming scores/probabilities of greater than 50%.

Other examples of models include but are not limited to Decision Tree, Linear Disciminant Analysis, Neural Network, Support Vector Machine, and k-Nearest Neighbor Classifier. In certain embodiments, a scoring algorithm comprises a method selected from the group consisting of: Linear Discriminate Analysis (LDA), Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naive Bayes, Naive Bayes Simple, Naive Bayes Up, IB1, Ibk, Kstar, LWL, AdaBoost, ClassViaRegression, Decorate, Multiclass Classifier, Random Committee, j48, LMT, NBTree, Part, Random Forest, Ordinal Classifier, Sparse Linear Programming (SPLP), Sparse Logistic Regression (SPLR), Elastic NET, Support Vector Machine, Prediction of Residual Error Sum of Squares (PRESS), and combinations thereof. A person of ordinary skill in the art could use there different models to evaluate expression level data and comparative data involving expression levels of one or more miRs (or their precursors or their targets). In some embodiments, the underlying classification algorithm is linear discriminate analysis (LDA). LDA has been extensively studied in the machine learning literature, for example, Hastie et al. (2009) and Venables & Ripley (2002), which are both incorporated by reference.

Models may take into account one or more diff pair values or they may also take into account differential expression of one or more miRNAs not specifically as part of a diff pair. A diagnostic or risk score may be based on 1, 2, 3, 4, 5, 6, 7, 8 or more diff pair values (or any range derivable therein), but in some embodiments, it takes into account additionally or alternatively, 1, 2, 3, 4, 5, 6, 7, 8, 9, 10 or more miRNA expression levels (or any range derivable therein), wherein the miRNA expression level detectably differs between PDAC cells and cells that are not PDAC.

In some embodiments, a score is prepared. The score may involve numbers such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, (or any range or a subset therein) in some embodiments.

### III. GNAS & KRAS

KRAS mutations at codon 12 (G12D, G12V, or G12R) have been identified in most PDACs as well as in 40 to 84% of IPMNs (Wu et al. SciTranal Med (2011); C. Almoguera, et al. Cell 53, 549-554 (1988); S. Fritz, et al., Ann. Surg. 249, 440-447 (2009); D. Soldini, et al. J. Pathol. 199, 453-461 (2003); F. Schönleben et al. Cancer Lett. 249, 242-248 (2007); K. Wada, et al. J. Gastrointest. Surg. 8, 289-296 (2004); S. Jones, et al. Science 321, 1801-1806 (2008)).

KRAS mutations at codon 13 have also been associated with malignancy in cystic tumors of the pancreas (Bartsch et. al Ann Surg 228(1): 79-86 (1998)).

GNAS mutations have been discovered recently and shown to play a driving role in the IPMN-specific pathway to pancreatic cancer (Wu et al. SciTransl Med (2011). These mutations occur at a single codon (201), endowing cells with extremely high adenylcyclase activity and adenosine 3′,5′-monophosphate(cAMP) levels (A. Diaz, et al J. Pediatr. Endocrinol. Metab. 20, 853-880 (2007); A. Lania, et al. Horm. Res. 71 (Suppl. 2), 95-100 (2009); A. G. Lania, et al. Nat. Clin. Pract. Endocrinol. Metab. 2, 681-693 (2006)).

The most important clinical utility for the combination of KRAS and GNAS mutations involves distinction with high sensitivity and specificity between SCA and mucinous cystic lesions (IPMN and MCN). In the most recent study from Wu et al. (2011) most IPMNs had a GNAS and/or a KRAS, while no SCAs had either mutation. in addition, the presence of a GNAS mutation in cyst fluid could also distinguish IPMNs from MCNs, although with a lower sensitivity.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The diagnostic benefit of using miRNAs expression changes and DNA mutations as biomarkers was assessed in resected pancreatic FFPE tissue, focusing on distinguishing benign from pre-malignant pancreatic cystic neoplasms and malignant pancreatic lesions. In particular, biomarkers allowing differentiation pre-malignant mucinous cystic neoplasms from branch duct intraductal papillary mucinous neoplasm (IPMN) were of particular interest. In brief, total nucleic acid was extracted from 69 macrodissected FFPE specimens, including serous cystadenoma (SN), branch duct IPMN (BD-IPMN), main duct IPMN (MD-IPMN), mucinous cystic neoplasm (MCN) and pancreatic ductal adenocarcinoma (PDAC).

Expression profiling of 377 miRNAs was performed using TaqMan MicroRNA Arrays Pool A in 30 specimens, including 5 PDAC, 5 SN, 10 MD-IPMN, 5 BD-IPMN, 5 MCN specimens. Verification of the top candidate miRNAs was performed using TaqMan MicroRNA Assays in all 69 FFPE specimens. This study identified a set of 27 differentially expressed miRNAs along with 3 potential miRNA normalizers (miRs-181-5p, -324-5p, and -345-5p), which distinguished patients with SN, MCN, PDAC, and IPMN, 6 additional miRNA identified in the previous studies were manually added to the study (miR-30a-3p, miR-342-3p, miR-93-5p, and miR-99a-5p, miR-24-3p, miR-375).

Logistic regression models based on 13 of the 27 differentially expressed miRNA species were capable of classifying: (1) MCN vs. branch duct IPMN (BD IPMN) with estimated 100% accuracy, (2) MCN vs. the merged set of SNs, PDACs, and IPMNs with estimated 100% accuracy, (3) SN vs. the merged set of MCNs. PDACs, and IPMNs with estimated 95% accuracy, and (4) PDAC vs. IPMN with estimated accuracy of 84%.

In addition, mutational status in KRAS codon 12/13 and GNAS codon 201 was interrogated via targeted resequencing or, the Ion Torrent's Personal Genome Machine (POM).

### Example 1—Methods

Patients and Biospecimens.

This study was approved by the Brigham and Women's Hospital (BWH) Institutional Review Board. The BWH Surgical Pathology Database was used to identify 69 formalin-fixed, paraffin-embedded (FFPE) tissue specimens of patients who underwent pancreatectomy for IPMN at the Brigham and Women's Hospital. This specimen set was composed of 20 PDAC, 20 SN, 10 MD IPMN, 10 BD IPMN and 9 MCN specimens, all confirmed by surgical pathology. For each of the specimens 1 encircled H&E slide and 10×4 μm unstained tissue slides were provided. The compilation of the specimens and diagnoses is provided in Table 1.

Histologic diagnoses were confirmed according to the latest World Health Organization recommendations (WHO) (Bosman et al., 2010). A consensus was reached in all cases.

Specimen Macrodissection.

Manual macrodissection was used to enrich for lesional tissue prior to total RNA extraction and molecular analysis. For the majority of the diagnostic categories, lesional tissue is epithelial. In brief, one H&E slide and up to 10 unstained slides were generated from each FFPE block. The H&E-stained glass slide was reviewed by a gastrointestinal pathologist, who used a marking pen to encircle the target lesion. Subsequently, the corresponding unstained slides were aligned with the H&E stained slide using sample edges and sample features. Non-target tissues (e.g. non-neoplastic pancreatic acinar, ductal, and endocrine tissue) were removed by incising along the circle, and scraping away the undesired areas. The remaining target tissue area was then available for total RNA extraction.

Total Nucleic Acid Extraction from Macrodissected FFPE Tissue.

Total nucleic acid (tNA), comprised of RNA, including (small RNA) and DNA was extracted from macrodissected FFPE tissues using the RecoverAll™ Total Nucleic Acid Isolation Kit for FFPE (Ambion/Life Technologies, Austin, Tex.) according to the manufacturer's protocol. This method allows robust and reproducible recovery of nucleic acid from FFPE tissues in sufficient quality and quantity to support mRNA, miRNA and DNA expression profiling studies (Doleshal et al., 2008). A part of the nucleic acid eluate was digested with DNase to allow focused recovery of total RNA for Megaplex high throughput miRNA expression analysis. The concentration and purity of both tNA and tRNA were assessed with a NanoDrop 1000 spectrophotometer (NanoDrop Technologies/Thermo Scientific, Wilmington, Del.).

The average tNA and tRNA recovery from the 69 macrodissected FFPE specimens were 11,514.5 ng (range: 697.4-48,076.6 ng) and 1,569 ng (range: 485-2,561 ng) (Table 1).

TABLE 1
Compilation of the 69 FFPE tissue samples used in this study, including their
diagnoses and the nucleic acid recovery.

| ASU ID | BWH ID | ng/ul | A260 | A280 | 260/280 | 260/230 | Total ng | Diagnosis |
|---|---|---|---|---|---|---|---|---|
| S0058077 | A1 | 107.43 | 2.149 | 1.125 | 1.91 | 1.43 | 11817.3 | SN |
| S0058078 | A2 | 116.15 | 2.323 | 1.18 | 1.97 | 1.8 | 12776.5 | SN |
| S0058079 | A3 | 30.01 | 0.6 | 0.343 | 1.75 | 1.47 | 3301.1 | SN |
| S0058080 | A4 | 48.01 | 0.96 | 0.501 | 1.92 | 1.48 | 5281.1 | SN |
| S0058081 | A5 | 64.44 | 1.289 | 0.652 | 1.98 | 1.35 | 7088.4 | SN |
| S0058082 | A6 | 201.62 | 4.032 | 2.009 | 2.01 | 1.65 | 22178.2 | SN |
| S0058083 | A7 | 70.67 | 1.413 | 0.716 | 1.97 | 1.55 | 7773.7 | SN |
| S0058084 | A8 | 14.23 | 0.285 | 0.172 | 1.66 | 0.96 | 1565.3 | SN |
| S0058085 | A9 | 132.97 | 2.659 | 1.317 | 2.02 | 1.33 | 14626.7 | SN |
| S0058086 | A10 | 16.6 | 0.332 | 0.187 | 1.77 | 1.21 | 1826 | SN |
| S0058087 | A11 | 30.24 | 0.605 | 0.33 | 1.83 | 1.57 | 3326.4 | SN |
| S0058088 | A12 | 16.55 | 0.331 | 0.188 | 1.76 | 1.73 | 1820.5 | SN |
| S0058089 | A13 | 81.75 | 1.635 | 0.834 | 1.96 | 1.61 | 8992.5 | SN |
| S0058090 | A14 | 115.16 | 2.303 | 1.164 | 1.98 | 1.82 | 12667.6 | SN |
| S0058091 | A15 | 143.46 | 2.869 | 1.399 | 2.05 | 1.89 | 15780.6 | SN |
| S0058092 | A16 | 17.18 | 0.344 | 0.19 | 1.81 | 1.54 | 1889.8 | SN |
| S0058093 | A17 | 115.9 | 2.318 | 1.159 | 2 | 1.55 | 12749 | SN |
| S0058094 | A18 | 131.04 | 2.621 | 1.292 | 2.03 | 1.92 | 14414.4 | SN |
| S0058095 | A19 | 160.49 | 3.21 | 1.682 | 1.91 | 1.2 | 17653.9 | SN |
| S0058096 | A20 | 151.43 | 3.029 | 1.549 | 1.96 | 1.44 | 16657.3 | SN |
| S0058097 | B1 | 70.79 | 1.416 | 0.728 | 1.94 | 1.46 | 7786.9 | MCN |
| S0058098 | B2 | 437.06 | 8.741 | 4.494 | 1.95 | 2.03 | 48076.6 | MCN |
| S0058099 | B3 | 60.87 | 1.217 | 0.599 | 2.03 | 1.8 | 6695.7 | MCN |
| S0058100 | B4 | 145.94 | 2.919 | 1.429 | 2.04 | 1.93 | 16053.4 | MCN |
| S0058101 | B5 | 26.44 | 0.529 | 0.274 | 1.93 | 1.64 | 2908.4 | MCN |
| S0058102 | B6 | 44.13 | 0.883 | 0.482 | 1.83 | 1.41 | 4854.3 | MCN |
| S0058103 | B7 | 46.99 | 0.94 | 0.496 | 1.89 | 1.51 | 5168.9 | MCN |
| S0058104 | B8 | 141.78 | 2.836 | 1.387 | 2.04 | 1.96 | 15595.8 | MCN |
| S0058105 | B9 | 121.18 | 2.424 | 1.232 | 1.97 | 1.78 | 13329.8 | MCN |
| S0058106 | B10 | 123.6 | 2.472 | 1.216 | 2.03 | 1.94 | 13596 | MCN |
| S0058107 | C1 | 40.02 | 0.8 | 0.421 | 1.9 | 1.57 | 4402.2 | PD PDAC |
| S0058108 | C2 | 57.81 | 1.156 | 0.624 | 1.85 | 1.51 | 6359.1 | PD PDAC |
| S0058109 | C4 | 105.03 | 2.101 | 1.074 | 1.96 | 1.66 | 11553.3 | PD PDAC |
| S0058110 | C5 | 12.29 | 0.246 | 0.142 | 1.73 | 1.39 | 1351.9 | PD PDAC |
| S0058111 | C6 | 44.39 | 0.888 | 0.5 | 1.77 | 1.26 | 4882.9 | PD PDAC |
| S0058112 | C7 | 46.96 | 0.939 | 0.485 | 1.94 | 1.56 | 5165.6 | PD PDAC |
| S0058113 | C8 | 186.86 | 3.737 | 1.859 | 2.01 | 1.67 | 20554.6 | PD PDAC |
| S0058114 | C9 | 123.16 | 2.463 | 1.223 | 2.01 | 1.81 | 13547.6 | PD PDAC |
| S0058115 | C10 | 75.37 | 1.507 | 0.774 | 1.95 | 1.33 | 8290.7 | PD PDAC |
| S0058116 | C11 | 39.12 | 0.782 | 0.423 | 1.85 | 1.31 | 4303.2 | PD PDAC |
| S0058117 | C12 | 60.21 | 1.204 | 0.613 | 1.97 | 1.24 | 6623.1 | PD PDAC |
| S0058118 | C13 | 191.59 | 3.832 | 1.877 | 2.04 | 1.81 | 21074.9 | PD PDAC |
| S0058119 | C14 | 189.91 | 3.798 | 1.912 | 1.99 | 1.7 | 20890.1 | PD PDAC |
| S0058120 | C15 | 73.29 | 1.466 | 0.745 | 1.97 | 1.73 | 8061.9 | PD PDAC |
| S0058121 | C16 | 135.55 | 2.711 | 1.346 | 2.01 | 1.55 | 14910.5 | PD PDAC |
| S0058122 | C17 | 86.2 | 1.724 | 0.884 | 1.95 | 1.66 | 9482 | PD PDAC |
| S0058123 | C18 | 141.31 | 2.826 | 1.397 | 2.02 | 1.78 | 15544.1 | PD PDAC |
| S0058124 | C19 | 79.29 | 1.586 | 0.797 | 1.99 | 1.54 | 8721.9 | PD PDAC |
| S0058125 | C20 | 24.43 | 0.489 | 0.299 | 1.64 | 0.74 | 2687.3 | PD PDAC |
| S0058126 | D1 | 173.32 | 3.466 | 1.76 | 1.97 | 1.65 | 19065.2 | BD-IPMN |
| S0058127 | D2 | 54.47 | 1.089 | 0.521 | 2.09 | 1.7 | 5991.7 | BD-IPMN |
| S0058128 | D3 | 386.92 | 7.738 | 4.122 | 1.88 | 1.4 | 42561.2 | BD-IPMN |
| S0058129 | D4 | 47.72 | 0.954 | 0.502 | 1.9 | 1.53 | 5249.2 | BD-IPMN |
| S0058130 | D5 | 60.02 | 1.2 | 0.61 | 1.97 | 1.78 | 6602.2 | BD-IPMN |
| S0058131 | D6 | 249.56 | 4.991 | 2.438 | 2.05 | 2.01 | 27451.6 | BD-IPMN |
| S0058132 | D7 | 113.93 | 2.279 | 1.117 | 2.04 | 2.09 | 12532.3 | BD-IPMN |
| S0058133 | D8 | 49.23 | 0.985 | 0.525 | 1.87 | 1.63 | 5415.3 | BD-IPMN |
| S0058134 | D9 | 75.15 | 1.503 | 0.767 | 1.96 | 1.65 | 8266.5 | BD-IPMN |
| S0058135 | D10 | 63.32 | 1.266 | 0.639 | 1.98 | 1.46 | 6965.2 | BD-IPMN |
| S0058136 | E1 | 331.58 | 6.632 | 3.221 | 2.06 | 2.07 | 36473.8 | MD-IPMN |
| S0058137 | E2 | 120.43 | 2.409 | 1.185 | 2.03 | 1.36 | 13247.3 | MD-IPMN |
| S0058138 | E3 | 144.49 | 2.89 | 1.432 | 2.02 | 1.51 | 15893.9 | MD-IPMN |
| S0058139 | E4 | 6.34 | 0.127 | 0.076 | 1.68 | 0.96 | 697.4 | MD-IPMN |
| S0058140 | E5 | 6.94 | 0.139 | 0.061 | 2.28 | 2.69 | 763.4 | MD-IPMN |
| S0058141 | E6 | 17.64 | 0.353 | 0.18 | 1.96 | 2 | 1940.4 | MD-IPMN |
| S0058142 | E7 | 279.03 | 5.581 | 2.711 | 2.06 | 2.1 | 30693.3 | MD-IPMN |
| S0058143 | E8 | 148.66 | 2.973 | 1.487 | 2 | 1.9 | 16352.6 | MD-IPMN |
| S0058144 | E9 | 84.94 | 1.699 | 0.836 | 2.03 | 1.75 | 9343.4 | MD-IPMN |
| S0058145 | E10 | 111.93 | 2.239 | 1.104 | 2.03 | 1.73 | 12312.3 | MD-IPMN |

Legend:
SN—serous cystadenoma,
MCN—mucinous cystic neoplasm,
PD PDAC—poorly differentiated PDAC,
BD-IMN—branch duct IPMN,
MD-IPMN: main duct IPMN (containing also mixed type = MD + BD)).

MIRNA Expression Analyses in 30 FFPE Specimens.

High-throughput (HT) miRNA expression analyses were performed to identify miRNAs that distinguish 1/MCN vs BD-IPMN, 2/MCN vs SN+PDAC+IPMN, 3/SN vs MCN+PDAC+IPMN and 4/PDAC vs MD IPMN. Expression levels of 377 mature miRNAs (Pool A) were interrogated using TaqMan MicroRNA Arrays in 5 PDAC, 5 SN, 10 MD-IPMN, 5 BD-IPMN and 5 MCN specimens. 10 ng of total RNA (tRNA) was converted into cDNA using Megaplex RT Primers (Applied Biosystems) and TaqMan miRNA RT Kits (Applied Biosystems). cDNA was pre-amplified (12 cycles) using Megaplex PreAmp Primers Pool A prior to mixing with TaqMan Universal PCR Master Mix (Applied Biosystems) and loading onto TaqMan human miRNA fluidic cards (Applied Biosystems). The cards were run using the Applied Biosystems 7900HT real-time PCR instrument equipped with a heating block for the fluidic card (Applied Biosystems). Prior to bioinformatics analysis, raw data were processed using Relative Quantification (ΔΔCt) and the RQ Manager, with baseline set to “automatic” and Ct threshold set to 0.2.

Bioinformatics Analysis of Megaplex miRNA Expression Data and Selection of Candidates.

Analysis of the Megaplex miRNA expression data was performed using a DiffPair normalization strategy. All pairwise combinations of a filtered set of miRNAs we taken as the basis of DiffPair biomarkers associated with the difference in Ct vetoes (ΔCt) between the two miRNA biomarkers composing the DiffPair. The filtered act of miRNA species consisted of those miRNA for which: (1) Ct values for all samples were net higher than 40, (2) the mean Ct value across all tested samples was below 30 (indicating reasonably robust expression levels), and (3) the standard deviation of Ct levels across all samples was above 1 (application of such overall variance-filtering strategies has been shown to increase detection power for high-throughput experiment (Bourgon, 2010)). Those miRNAs were incorporated into DiffPairs of hypothesis testing. Candidate miRNA species were selected by t-test and ANOVA analysis of the DiffPaired Megaplex Data for differential expression comparing: (1) BD IPMN vs. MCN, (2) BD IPMN vs. SN, (3) SN vs. MCN, and (4) PDAC vs. SN vs. all mucinous lesions pooled together (MCN+BD IPMN+MD IPMN), A single MD IPMN specimen (S0058139, E4) was removed from the set of 30 samples tested by Megaplex before performing this analysis due to a very large number of missing Ct values. In selecting candidate miRNA for further investigation, we applied different FDR p-value and log-ratio (log-base-two of fold change) cutoffs for different comparisons as a result of the very large number of significant miRNA found for some comparisons compared to others. The specific cutoffs applied we (1) fir BD IMPN vs. MCN, FDR<0.01, |log-ratio|>4.5; (2) for BD IPMN vs. SN, FDR<4.00125, |log-ratio|>6.5; (3) for SN vs. MCN. FDR≤0.00125, |log-ratio|>6.5; and (4) for PDAC vs. SN vs. mucinous, FDR≤1.25E-07, no log-ratio threshold.

TABLE 2
List of 35 Selected miRs based on the Megaplex miRNA
expression data and published data.

| | | | | |
|---|---|---|---|---|
| miR-10b-5p | miR-192-5p | miR-24-3p | miR-345-5p | miR-485-3p |
| miR-125a-3p | miR-200b-3p | miR-30a-3p | miR-363-3p | miR-489 |
| miR-130b-3p | miR-202-3p | miR-31-5p | miR-375 | miR-708-5p |
| miR-134 | miR-203 | miR-323a-3p | miR-379-5p | miR-885-5p |
| miR-135a-5p | miR-21-5p | miR-324-5p | miR-382-5p | miR-93-5p |
| miR-135b-5p | miR-210 | miR-337-5p | miR-429 | miR-98 |
| miR-181a-5p | miR-224-5p | miR-342-3p | miR-483-5p | miR-99a-5p |

Singleplex RT-qPCR verification of the top 35 miRNA candidates selected (see Table 2) was performed in the complete 69 FFPE specimen set. 10 ng total tRNA was used per reverse transcription reaction (30 min, 16*C: 30 min, 42° C.; 5 min, 85° C.; hold at 4° C.). Positive tissue QC and no-template control (NTC, nuclease-free water) samples were used to control for reagent performance and contamination. qPCR was run on the 7900HT instrument as follows: 10 min at 95° C.; 45 cycles of: 15 sec at 95° C. and 30 sec at 60° C.

Bioinformatic Analyses of Singleplex RT-qPCR Data.

For each sample, a normalization factor computed as the mean of the Ct values for the 3 Megaplex-selected normalizer miRNA species (miR-181a-5p, miR-324-5p, and miR-345-5p) was subtracted from the remaining 32 singleplex miRNA candidates to yield normalized expression values. Ten different pairwise comparisons were then tested for differential expression using t-tests. Benjamini-Hochberg false discovery rate adjustment was applied using 381 miRNA species tested by Megaplex (since this sample set overlapped with the Megaplex sample set). while Bonferroni correction was applied through application of a 0.005 FDR threshold to account for the 10 distinct pairwise comparisons being tested.

An L2-penalized logistic regression modeling strategy was employed using a modified stepwise feature selection procedure to construct models for each of four pairwise comparisons of interest: (1) MCN vs. BD IPMN, (2) MCN vs. all other conditions (SN/PDAC/BD IPMN/MD IPMN), (3) SN vs. all other conditions (MCN/PDAC/BD IMPN/MD IMPN), and (4) PDAC vs. IPMN (BD IPMN/MD IPMN).

MicroRNA Expression-Based Diagnostic Models.

A variety of different models can be employed to evaluate expression levels and/or other comparative values based on expression levels of miRNAs (or their precursors or targets). In particular, a logistic regression model (see the Wikipedia entry on the World Wide Web at en.wikipedia.org/wiki/Logistic_regression, which is hereby incorporated by reference) distinguishing between two diagnostic groups consists of a set of predictor variables, Xᵢ for i between 1 and n together with a set of weight coefficients wᵢ for i between 0 and n, from which the probability that a sample with predictor values Xᵢ=xᵢ is in the first diagnostic group can be computed as

$${p}_{malignant} = \frac{1}{1 + exp ( - {w}_{0} - {\sum }_{i = 1}^{n} {w}_{i} {x}_{i} )}$$

Other examples of models include but are not limited to decision trees, linear or quadratic discriminant analysis, neural networks, support vector machines, and k-nearest neighbor classifiers. A person of ordinary skill in the art could use these different modeling procedures to evaluate expression level data and comparative data involving expression levels of one or miRNAs (or their precursors or their targets).

Because of the difficulties involved in precisely controlling the amount of intact RNA input for qRT-PCR assays, it is generally desirable to construct models which take as inputs comparative differences in expression between two or more biomarkers instead of the raw cycle threshold (Ct) values measured for individual miRNA biomarkers. One method for accomplishing this is to consider a DiffPair consisting of two biomarkers A and B associated with the value computed as the difference in Ct value between marker A and marker B (i.e, if x_{A} is the Ct value of marker A and x_{B} is the Ct value of marker B, then x_{A}-x_{B} is the value of the DiffPair Diff(A,B)).

In fitting logistic regression models, an alternative method for adjusting for potential differential intact RNA input levels to the DiffPair method described above is to constrain the sum of the weight coefficients wᵢ for i >0 to be equal to zero:

$${\sum }_{i = 1}^{n} {w}_{i} = 0$$

The result of fitting such as a constrained logistic regression model is that, as in the case with DiffPair values, the model output scores are insensitive to any changes to biomarker Ct values that change the measured Ct values of all predictors upwards or downwards by the same amount, so that only relative expression levels between multiple biomarkers are used by the resulting model to predict the probability of malignancy. Note that in the case of exactly two biomarkers, this constrained logistic regression model becomes a logistic regression model built on a single DiffPair. More generally, models built with this type of constraint can be equivalently described in terms of an unconstrained logistic regression model built using a set of DiffPairs for more than two biomarkers as well (although this may increase the complexity of modeling process).

The classifier algorithms presented below were constructed using only subsets of the singleplex-measured miRNA biomarkers. For each model, the strategy for selection of the miRNA subset used by that model was to choose the first three biomarkers using an unconstrained L2-penalized stepwise logistic regression strategy (L2 penalty parameter set in all cases to λ₂=2.5). A fourth miRNA biomarker was chosen as the remaining biomarker with the most negative correlation with the mean expression level of the previously chosen biomarkers. This was done to ensure that the relative expression differences between biomarkers used by the final constrained logistic regression models to classify samples were of suitably robust magnitude: the signal consisting of the difference between the expression levels of an up-regulated and a down-regulated biomarker will have a greater magnitude difference (ΔΔCt) between diagnostic groupings than will the difference between two similarly up-regulated biomarkers, and hence is likely to be more robust in the presence of noise.

Once the subset of biomarkers to be used as predictors for a given model had been identified, the final model was constructed by fitting using the constrained logistic regression described above (again using an L2-penalty λ2=2.5) to the selected predictors. Classifier performance was estimated using leave-one-out-cross-validation evaluating the entire modeling process, including feature selection: only the cross-validation predictions made for those samples which were not tested by Megaplex were considered in estimating performance, so as to avoid statistical bias from the initial round of Megaplex-based feature reduction.

Mutational Analysis of KRAS Codon 12/13 and GNAS Codon 201.

Sample preparation of the custom next generation sequencing (NGS) panel comprised 5 steps: 1) gene-Specific PCR, 2) tag PCR, 3) library pooling, 4) purification and 5) library quantification and dilution. In short, DNA isolated from 68 specimens (1 FFPE specimen, S0058139 (E4), was exhausted during the initial miRNA candidate discovery and verification) was quantified via NanoDrop (ND-1000) to establish concentration, yield and purity and normalized to 5 ng/μL. A PCR-based approach enriched for KRAS (codons 4-15) and GNAS (codon 201) from 10 ng DNA using 30 cycles of targeted gene-specific PCR, followed by sample barcoding using 10 cycles of Tag PCR. The FAM-labeled amplicons were analyzed by capillary electrophoresis (CE). A procedural no-template control (NTC) and an admixed cancer cell-line mixture (2-35%) were included. Individual Fragment libraries were pooled, column-purified, eluted according to manufacturers' guidelines (QIAGEN) and quantified on the Agilent 2100 Bioanalyzer to assess concentration and ensure proper sizing distribution (in bp). The pooled library was diluted to 50 pM (30×10⁶ copies/μL) prior to performing emPCR with 150 million copies input using Ion Torrent's Personal Genome Machine (PGM) system (Ion One Touch, ES and PGM). Pre-processing of the PGM sequence data was accomplished during the following steps: filtering by Q17 quality, splitting samples by barcode, trimming barcode, adaptor and primer, followed by sequence alignment. Pre-processed and primer trimmed reads were processed using NextGENe® v2.1.8 or v2.2.0 (Softgenetics®). Reads were aligned by amplicon (to report coverage) and/or by gene (to report mutation calls) with the following alignment criteria: allowable mismatched bases=2, ≥90% of the read must match to the reference sequence. Coverage was assessed per amplicon by the number of aligned sequence reads to the amplicon reference. Mutation positive calls were reported from the filtered and aligned data at positions with a Mean Allele Frequency (MAF) of ≥5%.

### Example 2—Results

miRNA Candidate Discovery.

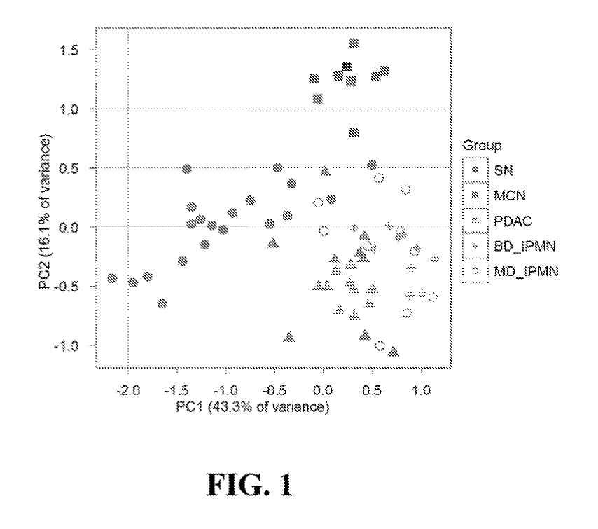

Initial expression profiling of 377 mature miRNAs was performed in 30 macrodissected FFPE specimens comprising SN (n=5), PDAC (n=5), BD-IPMN (n=5), MD-IPMN (n=5) and MCN (n=5) (Table 1). Use of multiplex RT and cDNA pre-amplification allowed significant reduction of the tRNA input relative to singleplex RT-qPCR. Data from Asuragen (unpublished) and other research groups show that pre-amplification of miRNA-containing cDNA improves sensitivity of miRNA detection, while maintaining the relative expression levels (Mestdagh et al., 2008; Chen et al., 2009). Clear separation between experimental groups was observed (FIG. 1). The bioinformatics analysis focused on the 4 most important comparisons produced 38 unique DiffPairs composed of 30 unique miRNAs, including: miR-10b-5p, miR-21-5p, miR-31-5p, miR-98, miR-125a-3p, miR-130b-3p, miR-134, miR-135a-5p, miR-135b-5p, miR-192-5p, miR-194-5p, miR-200a-3p, miR-200b-3p, miR-200c-3p, miR-202-3p, miR-203, miR-210, miR-224-5p, miR-323a-3p, miR-337-5p, miR-345-5p, miR-363-3p, miR-379-5p, miR-382-5p, miR-429, miR-483-5p, miR-483-3p, miR-489, miR-708-5p, miR-8S-5p. Table 3 contains FDR adjusted p-values for 38 selected DiffPairs. Those FDR values below the comparison-specific cutoffs used in selecting these DiffPairs (described in methods above) are highlighted in gray.

3 of the 30 miRNA candidates identified through analysis of the Megaplex data were removed due to likely redundancy identified through consideration of the correlation of their expression profiles with other candidates: miR-194-5p was very highly correlated with miR-192-5p, while miR-200a-3p and miR-200c-3p were very highly correlated with miR-200b-3p. Analysis of p-values and mean expression level were used to select which miRNAs to retain and which to remove in these cases. 30 of the 38 originally selected DiffPairs did not contain any of these 3 eliminated candidates.

Two miRNAs (miR-181a-5p and miR-324-5p) were identified as potentially useful “normalizer” candidates from the Megaplex FFPE data because of high correlation with the mean expression level of all Megaplex-measured miRNA species for which expression could be consistently tested across all 30 samples (both concordance and Spearman correlation coefficients were considered). One of the 30 miRNA candidates identified by DiffPair analysis, miR-345-5p, was also identified as a potential normalizer in this manner.

In addition to 29 miRNA identified using Megaplex platform, 6 additional miRNA species were included as candidates because of identification in previous classifier projects: miR-30a-3p, miR-342-3p, miR-93-5p, and miR-99a-5p were identified as candidates in a study of pancreatic FFPE tissue samples (Matthaei et al., 2012); miR-24-3p (along with the afore-mentioned miRs-30a-3p and -342-3p) were identified as part of a classifier for pancreatic cyst fluid fine needle aspirate samples (Matthaei H. et al. Clinical Cancer Research 2012); and miR-375 was identified as part of a distinct pancreatic tissue classifier (methods describing pancreatic tissue classifier methods are discussed in U.S. patent application Ser. No. 13/615,066 incorporated herein by reference).

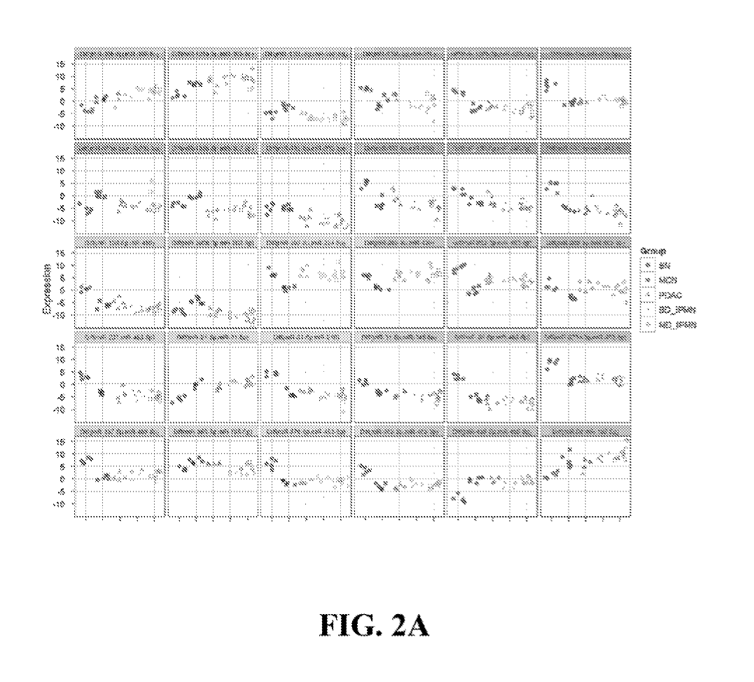

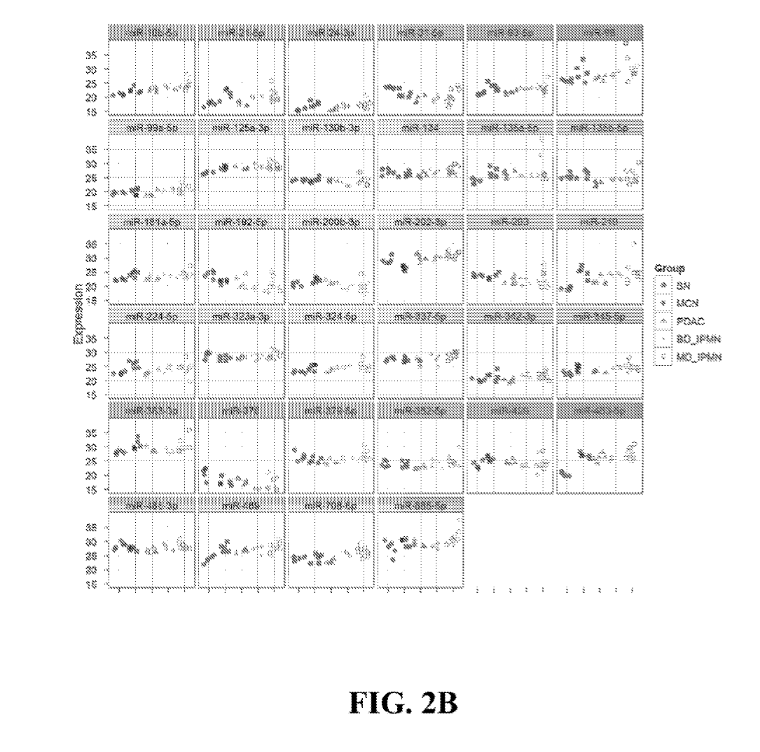

FIG. 2A reflects the Megaplex ΔCt values for the 30 differentially expressed DiffPairs remaining after elimination of miRs-194-5p, -200a-3p, and -200c-3p. FIG. 2B shows raw Megaplex Ct values for 34 miRNAs indicated for further verification with the singleplex RT-qPCR. miR-30a-3p, which was not tested by Megaplex, but was identified as a top candidate in a previous project, was added manually to the final miRNA set for a total of 35 miRNA candidates.

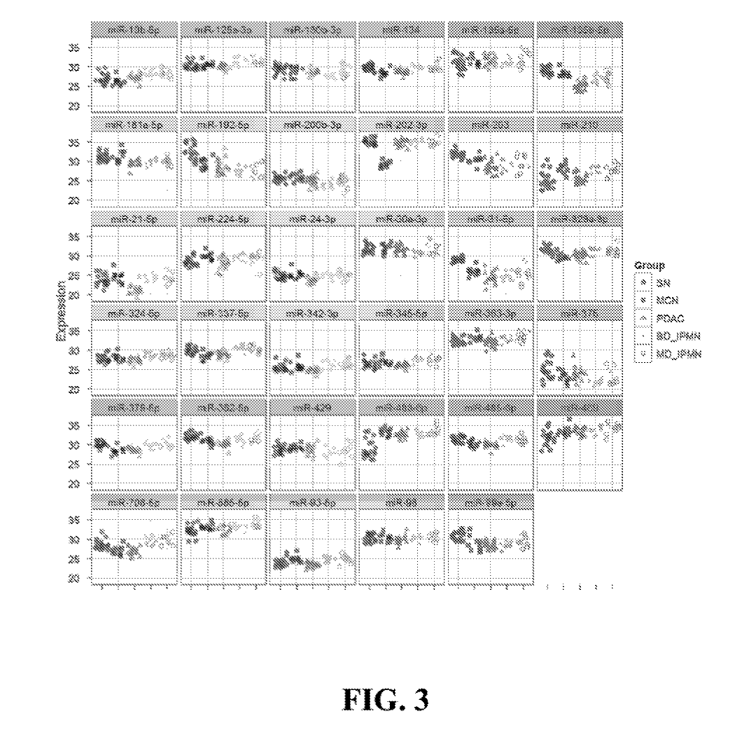

The 35 miRNA candidates selected after analysis of be Megaplex data set were then verified using singleplex RT-qPCR. After normalization using the 3 chosen miRNA normalizers, 27 of the remaining 32 candidates were significant (by t-test or ANOVA, depending on the comparison) at FDR<0.005 (significance level of 0.005 used to Bonferroni-correct for 10 distinct hypotheses being tested). The FDR values these analyses are shown in Table 4 (with the values below the significance threshold of 0.005 highlighted in gray); the Ct values for the individual miRs are shown in FIG. 3.

The intercept (w₀) and weight coefficients (w₁ for i>0) for the 4 constrained logistic regression classifiers trained as described in the methods section above are indicated in Table 3. The leave-one-out-cross-validation estimated accuracies and AUCs of the models for those samples tested by singleplex only were: (1) BD IPMN vs. MCN: accuracy 100% (95% CI: 69%-100%), AUC 1.0; (2) MCN vs. SN/PDAC/IPMN: accuracy 100% (95% CI: 91%-100%), AUC 1.0; (3) SN vs. MCN/PDAC/IPMN: accuracy 95% (95% CI: 83%-99%), AUC 0.99; and (4) PDAC vs. IPMN: accuracy 84% (95% CI: 60%-97%), AUC 0.93.

TABLE 5
Model coefficients.

| | MCN | | | PDAC |
|---|---|---|---|---|
| | vs. BD | MCN vs. | SN vs. | vs. |
| Predictor | IPMN | SN/PDAC/IPMN | MCN/PDAC/IPMN | IPMN |
| (Intercept) | 3.27 | −5.29 | 2.65 | 0.55 |
| miR-10b-5p | | 0.03 | | |
| miR-21-5p | | | | −0.41 |
| miR-31-5p | | | 0.80 | |
| miR-99a-5p | | | 0.49 | |
| miR-130b-3p | 0.37 | | | |
| miR-192-5p | 0.51 | | | |
| miR-202-3p | −0.80 | 1.09 | | |
| miR-210 | | −0.76 | | |
| miR-337-5p | −0.08 | | | |
| miR-375 | | −0.35 | −0.17 | 0.72 |
| miR-483-5p | | | −1.12 | |
| miR-485-3p | | | | 0.33 |
| miR-708-5p | | | | −0.63 |

Both of the models involving classification of MCN samples from (1) BD IPMN only and (2) SN/PDAC/IPMN together have miR-202-3p as their highest-weighted predictor. Table 5 shows that this miRNA appears to be highly upregulated in MCN compared to all other tested clinical groups. The expression of miR-202-3p is particularly contrasted with that of miRs-192-5p and -130b-3p in model (1) and with miRs-210 and -375 in model (2).

Model (3), SN vs. MCN/PDAC/IPMN, makes heaviest use of miRs-31-5p and -483-5p, which are down- and up-weighted, respectively, in most SN samples compared to the remaining clinical groups. MiR-99a-5p appears to supplement the signal of miR-31-5p in a similar manner in this model.

Model (4), PDAC vs. IPMN, appears to weight its predictors somewhat more evenly than the other models, perhaps because no one predictor seems to provide as clear of a signal. Both miR-375 and miR-708-5p are highly weighted, though in opposite directions since they are, respectively, down- and up-regulated in PDAC relative to IPMN, but they are not obviously much better than miR-21-5p on an individual miRNA level (Table 5).

Mutational Analysis of KRAS Codon 12/13 and GNAS Cotton 201.

The mutational status of KRAS codon 12/13 and GNAS codon 201 was interrogated in 68 FFPE specimens (excluding E5, due to exhaustion of material) via targeted resequencing on the Ion Torrent's Personal Genome Machine (PGM) as described in the Methods section. A cut-off of 3% was used to determine the presence of a given mutation. The raw sequencing data for KRAS and GNAS genes are compiled in Table 6, and summarized in Table 7. In the group of SN, 20% specimens (n=4/20) had a mutation (2 GNAS, 1 KRAS G12C, 1 KRAS G15D, no double mutations). In the group of MCNs, 10% specimens (n=10) had a mutation (KRAS G13D, no double mutations). In the PDAC group, 94.7% specimens (n=18/19) had a mutation (no GNAS, 7 KRAS G12V, 9 KRAS G12D, 1 KRAS G12S, 1 KRAS G12R, no double mutations). In the BD-IPMN group, 80% specimens (n=8/10) had a mutation and 4 double mutant specimens were uncovered (1 KRAS G12V, 1 KRAS G12D, 1 KRAS G12C, 1 GNAS R201H, 2 KRAS G12V/GNAS R201H, 1 KRAS G12D/GNAS R201H, 1 KRAS G12V/GNAS R201C). And finally, in the group of MD-IPMNs, 60% specimens (n=6/10) had a mutation and 4 double mutant specimens were uncovered (1 GNAS R201H, 1 GNAS R201C, 1 KRAS G12V/GNAS R201H, 2 KRAS G12D/GNAS R201H, 1 KRAS G2D/G12C). When BD- and MD-IPMN specimens were combined, 35% (n=7/20) contained both GNAS and KRAS mutation.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Olsen et al., 1999; Seggerson et al., 2002
Bartsch et. al Ann Surg 228(1): 79-86 (1998)
Denli et al., 2003
Froehler et al., 1986
Sambrook et al., 2001
Itakura et al., 1975
Gillam et al, 1978; Gillam et al., 1979
Klostermeier et al., 2002; Emptage, 2001; Didenko, 2001
Griffey et al., 1997
Cummins et al., 1996
Fodor et al., 1991
Hastie et al. (2009) and Venables & Ripley (2002)
Bosman et al., 2010
Doleshal et al., 2008
Mestdagh et al., 2008; Chen et al., 2009
Matthaei et al., 2012
Wu et al. Sci Transl Med (2011)
C. Almoguera, et al. Cell 53, 549-554 (1988)
S. Fritz, et al., Ann. Surg. 249, 440-447 (2009)
D. Soldini, et al. J. Pathol. 199, 453-461 (2003)
F. Schönleben et al. Cancer Lett. 249, 242-248 (2007)
K. Wada, et al. J. Gastrointest. Surg. 8, 289-296 (2004)
S. Jones, et al. Science 321, 1801-1806 (2008)
Bourgon R, Gentleman R, Huber W PNAS USA 2010 May 25; 107(21):9546-51.
U.S. Pat. No. 5,681,947
U.S. Pat. No. 5,652,099
U.S. Pat. No. 5,763,167

1-220. (canceled)

221. A method for treating a patient with mucinous cystic neoplasm (MCN), serous cystadenoma (SN), pancreatic ductal adenocarcinoma (PDAC), intraductal papillary mucinous neoplasm (IPMN), or a subtype thereof, the method comprising:
a) producing amplified labeled nucleic acid molecules that correspond to miR-202-3p, miR-483-5p, miR-31-5p, and miR-192-5p from a biological sample of a patient who has been determined to have pancreatic neoplastic cells
b) measuring the levels of expression of the amplified labeled nucleic acid molecules;
c) calculating a risk score based upon the measured levels of the amplified labeled nucleic acid molecules that identifies the sample as containing pancreatic cells that are characterized as mucinous cystic neoplasm (MCN), serous cystadenoma (SN), pancreatic ductal adenocarcinoma (PDAC), intraductal papillary mucinous neoplasm (IPMN), or a subtype thereof; and
d) treating the patient that has the biological sample that has been identified as having mucinous cystic neoplasm (MCN), serous cystadenoma (SN), pancreatic ductal adenocarcinoma (PDAC), intraductal papillary mucinous neoplasm (IPMN), or a subtype thereof with chemotherapy or radiation.

222. The method of claim 221, wherein the label is non-radioactive.

223. A composition comprising amplified labeled nucleic acid molecules that correspond to miR-202-3p, miR-483-5p, miR-31-5p, and miR-192-5p or at least one of the following diff pair miRNAs: miR-10b-5p, miR-21-5p, miR-31-5p, miR-98, miR-125-3p, miR-130b-3p, miR-134, miR-135a-5p, miR-135b-5p, miR-192-5p, miR-194-5p, miR-200a-3p, miR-200b-3p, miR-200c-3p, miR-202-3p, miR-203, miR-210, miR-224-5p, miR-323-3p, miR-337-5p, miR-345-5p, miR-363-3p, miR-379-5p, miR-382-5p, miR-429, miR-483-5p, miR-485-3p, miR-485-5p, miR-489, miR-708-5p, or miR-885-5p.

224. The composition of claim 223, wherein the label is non-radioactive.

225. A method for treating a patient with mucinous cystic neoplasm (MCN), serous cystadenoma (SN), pancreatic ductal adenocarcinoma (PDAC), intraductal papillary mucinous neoplasm (IPMN), or a subtype thereof, the method comprising:
a) producing amplified labeled nucleic acid molecules from a biological sample from the patient that correspond to at least one of the following diff pair miRNAs: miR-10b-5p, miR-21-5p, miR-31-5p, miR-98, miR-125-3p, miR-130b-3p, miR-134, miR-135a-5p, miR-135b-5p, miR-192-5p, miR-194-5p, miR-200a-3p, miR-200b-3p, miR-200c-3p, miR-202-3p, miR-203, miR-210, miR-224-5p, miR-323-3p, miR-337-5p, miR-345-5p, miR-363-3p, miR-379-5p, miR-382-5p, miR-429, miR-483-5p, miR-485-3p, miR-485-5p, miR-489, miR-708-5p, or miR-885-5p;
b) measuring the level of expression based upon the amplified labeled nucleic acid molecules of at least one of the following diff pair miRNAs: miR-10b-5p, miR-21-5p, miR-31-5p, miR-98, miR-125-3p, miR-130b-3p, miR-134, miR-135a-5p, miR-135b-5p, miR-192-5p, miR-194-5p, miR-200a-3p, miR-200b-3p, miR-200c-3p, miR-202-3p, miR-203, miR-210, miR-224-5p, miR-323-3p, miR-337-5p, miR-345-5p, miR-363-3p, miR-379-5p, miR-382-5p, miR-429, miR-483-5p, miR-485-3p, miR-485-5p, miR-489, miR-708-5p, or miR-885-5p, wherein at least one of the miRNAs is a biomarker miRNA and one is a comparative miRNA;
c) determining at least one biomarker diff pair value based on the level of expression of the biomarker miRNA compared to the level of expression of the comparative miRNA;
d) determining whether the neoplastic pancreatic cells are mucinous cystic neoplasm (MCN), serous cystadenoma (SN), pancreatic ductal adenocarcinoma (PDAC), intraductal papillary mucinous neoplasm (IPMN), or a subtype thereof, based on the biomarker diff pair value(s), and
e) treating the patient that has a neoplastic pancreatic cells that have been identified as having mucinous cystic neoplasm (MCN), serous cystadenoma (SN), pancreatic ductal adenocarcinoma (PDAC), intraductal papillary mucinous neoplasm (IPMN), or a subtype thereof with chemotherapy or radiation.

226. The method of claim 225, wherein the diff pair miRNAs comprises miR-130b-3p, miR-192-59, miR-202-3p, and miR-337-5p.

227. The method of claim 225, wherein the diff pair miRNAs comprises miR-10b-5p, miR-202-3p, miR-210, and miR-375.

228. The method of claim 225, wherein the diff pair miRNAs comprises miR-31-5p, miR-99a-5p, miR-375, and miR-483-5p.

229. The method of claim 225, wherein the diff pair miRNAs comprises miR-21-5p, miR-375, miR-485-3p, and miR-708-5p.
